(19) United States
(12) Patent Application Publication
(10) Pub. No.: US 2026/0176393 A1
(43) Pub. Date: Jun. 25, 2026

(54) PRESERVATIVE COMPOSITION FOR BIOLOGICAL SAMPLES

(71) Applicant: KANAZAWA UNIVERSITY, Kanazawa-shi (JP)

(72) Inventors: Kosuke KURODA, Kanazawa-shi (JP); Eishu HIRATA, Kanazawa-shi (JP); Daisuke TANAKA, Tsukuba-shi (JP)

(73) Assignee: KANAZAWA UNIVERSITY, Kanazawa-shi (JP)

(21) Appl. No.: 18/876,047

(22) PCT Filed: Jun. 16, 2023
(86) PCT No.: PCT/JP2023/022488

§ 371 Date: Dec. 17, 2024

(30) Foreign Application Priority Data
Jun. 17, 2022 (JP) 2022-098339
Mar. 6, 2023 (JP) 2023-034044

(51) Int. Cl. C08F 220/18; A61K 9/00; A61K 47/26; C08F 220/60

(52) U.S. Cl. CPC C08F 220/1804; A61K 9/0019; A61K 47/26; C08F 220/606

(57) ABSTRACT

A zwitterion polymer of formula (1) or a labeled form thereof, and a preservative composition for biological samples, comprising the same

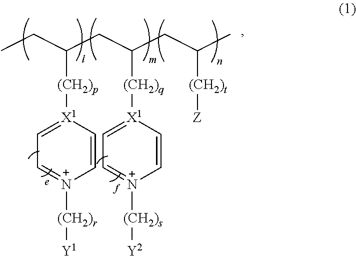

wherein X¹ and X² each represent a carbon or nitrogen atom; Y¹ and Y² each represent —COO⁻, —SO₃⁻, —OP═O(H)O⁻, —OP═O(CH₃)O⁻, or —OP═O(OR¹)O⁻; Z represents a hydrogen atom, an aromatic hydrocarbon group having 6 to 10 carbon atoms, an 5- or 6-membered aromatic heterocyclic group, an nitrogen-containing heterocyclic ammonium salt, a tetraalkylammonium salt, a tetraphenylphosphonium salt, a tetraalkylphosphonium salt, a trialkylsulfonium salt, or an alkyl group having 1 to 22 carbon atoms; e and f each represent an integer 0 or 1; 1, m, and n are each a number which satisfies 0<1≤1, 0≤m<1, 0≤n<1, and 1+m+n=1; and p, q, r, s, and t are each an integer 0 to 6.

### TECHNICAL FIELD

The present invention relates to a preservative composition for biological samples such as cells.

### BACKGROUND ART

Dimethyl sulfoxide (DMSO) and glycerol are widely used as cryopreservatives in cryopreserving cells and are the most effective reagents for protecting cells or organelles. These cryopreservatives protect cells by suppressing the growth of crystals of ice (ice crystals) formed in the cells at the time of cell freezing. DMSO and the like have cell membrane permeability and inhibit ice crystal formation by delaying the growth rate of ice crystals inside and outside cells. However, DMSO and glycerol have toxicity and are known to cause hypertension, nausea, and vomiting when transfused together with cells to a recipient or when handled by a person in charge of cells. In the case of using DMSO as a cryopreservative, fetal bovine serum (FBS) or bovine serum albumin (BSA) is often added therewith. These components disadvantageously vary in quality among lots because of being animal-derived. The present inventors have previously proposed an aprotic zwitterion as a substance which can be used instead of DMSO and the like commonly used as an additive component for media, and proposed a medium for cryopreservation and the like (Patent Literature 1).

In the case of adding a poorly soluble substance which is difficult to dissolve in water, to a medium in the assay of chemicals using cells, the poorly soluble substance is dissolved in a solvent such as DMSO and then dispersed in a medium. Although DMSO is assumed to be a solvent which offers very high solubility, it is known that some poorly soluble substances are not dissolved even in DMSO. The present inventors have previously proposed use of an aprotic zwitterion as a substance which dissolves a poorly soluble substance (Patent Literature 2).

Meanwhile, zwitterion polymers are used for modifying the surface of biocompatible polymers (Patent Literature 3) and also have been paid attention as industrial materials. Various studies have also been made on the structures and physical properties of zwitterions from an academic standpoint (Non Patent Literatures 1 to 3).

### CITATION LIST

### Patent Literature

Patent Literature 1: WO 2020/230721
Patent Literature 2: JP-A-2018-191623
Patent Literature 3: JP-A-2015-213755

### Non Patent Literature

Non Patent Literature 1: Eur. Polym. J. Vol. 26, No. 4, p. 415-421 (1990)
Non Patent Literature 2: ACS Appl. Mater. Interfaces 2018, 10, 17771-17783
Non Patent Literature 3: Chem Asian J. 2021, 16, 1897-1900

### SUMMARY OF INVENTION

### Technical Problem

The present inventors have previously produced an aprotic zwitterion as a substance which can be used as an alternative of DMSO, etc. commonly used as an additive component for media, and proposed that the aprotic zwitterion can be used in media for cryopreservation, solubilizers for poorly soluble substances, and the like. However, sufficiently effective have not been obtained.

It is an object of the present invention to provide a zwitterion polymer which can be used instead of DMSO and the like used as an additive component for biological samples and as a solubilizer for poorly soluble substances. It is also an object of the present invention to provide a preservative composition for biological samples, comprising such a polymer which is used in combination with a cell-penetrating substance, thereby reducing the toxicity of the cell-penetrating substance and further enhancing a cryopreserving effect.

### Solution to Problem

Accordingly, the present inventors have studied effects of various compounds on the cryopreservation of biological samples such as cells. As a result, the present inventors have found that when a zwitterion polymer having a specific structure is used, a water-soluble compound such as an electrolyte is added for elevating an osmotic pressure, and the solubility and dispersibility of the zwitterion polymer are adjusted, the preservability of biological samples such as cells directly or indirectly is improved. The present inventors have also found that a poorly soluble substance can be dissolved. Thus, the present invention has been accomplished.

The present invention provides the following items [1] to [15] of the invention.

[1] A zwitterion polymer of the following formula (1) or a labeled form thereof:

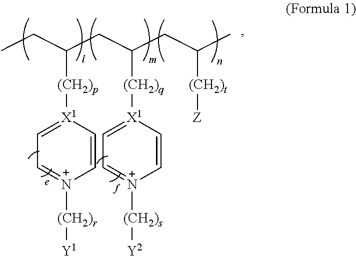

wherein
X¹ and X² are the same or different and each represent a carbon atom or a nitrogen atom;
Y¹ and Y² are the same or different and each represent an anion selected from the group consisting of —COO⁻, —SO₃⁻, —OP═O(H)O⁻, —OP═O(CH³)O⁻, and —OP═O(OR¹)O⁻;
Z represents a hydrogen atom, an optionally alkyl group-substituted aro hydrocarbon group having 6 to 10 carbon atoms, an optionally alkyl group-substituted 5- or 6-membered aromatic heterocyclic group, an optionally alkyl group-substituted nitrogen-containing heterocyclic ammonium salt, a tetraalkylammonium salt, a tetraphenylphosphonium salt, a tetraalkylphosphonium salt, a trialkylsulfonium salt, or a linear or branched alkyl group having 1 to 22 carbon atoms and optionally having 1 to 3 oxygen atoms in a molecular chain;
e and f each represent an integer of 0 or 1;
l, m, and n are each a number which indicates a content ratio of each repeat unit and represent a number which satisfies 0<l≤1, 0≤m<1, 0≤n<1, and l+m+n=1; and
p, q, r, s, and t each represent an integer of 0 to 6.

[2] The zwitterion polymer or the labeled form thereof according to [1], wherein Y¹ and Y² are the same or different and are each an anion selected from the group consisting of —COO⁻ and —SO₃⁻.

[3] The zwitterion polymer or the labeled form thereof according to [1] or [2], wherein Z is a group selected from the group consisting of an imidazolyl group, a pyridyl group, pyridinium chloride, C1 to C22 alkylpyridinium chloride, imidazolinium chloride, C1 to C22 alkylimidazolinium chloride, pyridinium bromide, C1 to C22 alkylpyridinium bromide, imidazolinium bromide, and C1 to C22 alkylimidazolinium bromide.

[4] A preservative composition for biological samples, comprising a zwitterion polymer of the following formula (1) or a labeled form thereof:

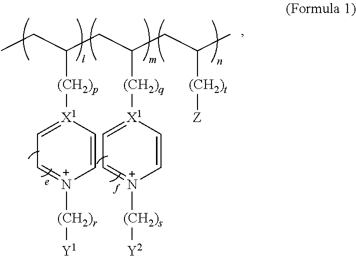

wherein
X¹ and X² are the same or different and each represent a carbon atom or a nitrogen atom;
Y¹ and Y² are the same or different and each represent an anion selected from the group consisting of —COO⁻, —SO₃⁻, —OP═O(H)O⁻, —OP═O(CH³)O⁻, and —OP═O(OR¹)O⁻;
Z represents a hydrogen atom, an optionally alkyl group-substituted aro hydrocarbon group having 6 to 10 carbon atoms, an optionally alkyl group-substituted 5- or 6-membered aromatic heterocyclic group, an optionally alkyl group-substituted nitrogen-containing heterocyclic ammonium salt, a tetraalkylammonium salt, a tetraphenylphosphonium salt, a tetraalkylphosphonium salt, a trialkylsulfonium salt, or a linear or branched alkyl group having 1 to 22 carbon atoms and optionally having 1 to 3 oxygen atoms in a molecular chain;
e and f each represent an integer of 0 or 1;
l, m, and n are each a number which indicates a content ratio of each repeat unit and represent a number which satisfies 0<l≤1, 0≤m<1, 0≤n<1, and l+m+n=1; and
p, q, r, s, and t each represent an integer of 0 to 6.

[5] The preservative composition for biological samples according to [4], wherein Y¹ and Y² are the same or different and are each an anion selected from the group consisting of —COO⁻ and —SO₃⁻.

[6] The preservative composition for biological samples according to [4] or [5], wherein Z is a group selected from the group consisting of an imidazolyl group, a pyridyl group, pyridinium chloride, a C1-C22 alkylpyridinium chloride, imidazolinium chloride, a C1-C22 alkylimidazolinium chloride, pyridinium bromide, a C1-C22 alkylpyridinium bromide, imidazolinium bromide, and a C1-C22 alkylimidazolinium bromide.

[7] The preservative composition for biological samples according to any one of [4] to [6], further comprising one or more water-soluble compounds selected from the group consisting of an electrolyte, betaine, a zwitterion, an alcohol, a polyhydric alcohol, and a saccharide.

[8] The preservative composition for biological samples according to any one of [4] to [7], further comprising a cell-penetrating substance.

[9] The preservative composition for biological samples according to any one of [4] to [8], wherein the preservative composition is a cryopreservative composition for biological samples, a composition for culture of biological samples, a medium composition for biological samples, a composition for preservation of biological samples, a composition for functional maintenance of biological samples, or a composition for a functional test of biological samples.

[10] A solubilizer for poorly soluble substances, comprising the zwitterion polymer or the labeled form thereof as defined in any one of [1] to [3].

[11] A method for preserving a biological sample, comprising contacting the biological sample with a composition comprising the zwitterion polymer or the labeled form thereof as defined in any one of [1] to [3].

[12] The method for preserving a biological sample according to [11], wherein the composition comprising the zwitterion polymer or the labeled form thereof as defined in any one of [1] to [3] further comprises one or more water-soluble compounds selected from the group consisting of an electrolyte, betaine, a zwitterion, an alcohol, a polyhydric alcohol, and a saccharide.

[13] The method for preserving a biological sample according to [11] or [12], wherein the composition comprising the zwitterion polymer or the labeled form thereof as defined in any one of [1] to [3] further comprises a cell-penetrating substance.

[14] The method for preserving a biological sample according to any one of [11] to [13], wherein the biological sample is preserved for cryopreservation of the biological sample, culture of the biological sample, a medium of the biological sample, functional maintenance of the biological sample, or a functional test of the biological sample.

[15] A method for dissolving a poorly soluble substance, comprising dissolving the poorly soluble substance in a composition comprising the zwitterion polymer or the labeled form thereof as defined in any one of [1] to [3].

### Advantageous Effects of Invention

By using the preservative composition for biological samples of the present invention, the survival rate after cryopreserving cells, etc. and then thawing is high, and biological sample such as cells can thereby be protected. Furthermore, by using the preservative composition of the present invention, the preservability, maintainability, etc. of biological materials are improved. Further, by using the zwitterion polymer of the present invention, various poorly soluble substances can be dissolved.

### BRIEF DESCRIPTION OF DRAWINGS

FIG. 1 shows a ¹H-NMR chart of VimC₃C (DMSO−).

FIG. 2 shows a ¹H-NMR chart of Poly(VimC₃C) (methanol).

FIG. 3 shows a ¹H-NMR chart of Poly(VimC₃C)+NaCl addition.

FIG. 4 shows a ¹H-NMR chart of Poly(VpyC₃C) (methanol-d6).

FIG. 5 shows a ¹H-NMR chart of Poly(VpyC₃S) (D₂O).

FIG. 6 shows a ¹H-NMR chart of VimC₃S (DMSO).

FIG. 7 shows a ¹H-NMR chart of Poly(VimC₃S) (D₂O).

FIG. 8 shows a ¹H-NMR chart of Poly(VpyC₃C)50 (CH₃OH-d).

FIG. 9 shows a ¹H-NMR chart of Poly(VpyC₃C)40 (CH₃OH-d).

FIG. 10 shows a ¹H-NMR chart of Poly(VpyC₃C)30 (CH₃OH-d).

FIG. 11 shows a ¹H-NMR chart of Poly(VpyC₃C)20 (CH₃OH-d).

FIG. 12 shows a ¹H-NMR chart of Poly(VpyC₃C)10 (CH₃OH-d).

FIG. 13 shows a ¹H-NMR chart of Poly(VpyC₃S)40 (methanol-NaCl).

FIG. 14 shows a 1H-NMR chart of Poly(VimC₃C-co-C₈Vim).

FIG. 15 shows the relationship between the concentration of the zwitterion polymer and the survival rate of cells after cryopreservation.

FIG. 16 shows the relationship between the concentration of an aqueous NaCl solution added to the zwitterion polymer and the survival rate of cells after cryopreservation.

FIG. 17 shows the relationship between different types of solutes added to the zwitterion polymer and the survival rate of cells after cryopreservation.

FIG. 18 shows the toxicity of the zwitterion polymer to cells.

FIG. 19 shows a DLS chart of a NaCl solution of the zwitterion polymer.

FIG. 20 shows GPC results of the zwitterion polymer.

FIG. 21 shows the relationship between a catalyst/monomer ratio at the time of zwitterion polymer production and the survival rate after cryopreservation. In the Fig., the left part depicts data on an aqueous zwitterion polymer solution, and the right part depicts data on a zwitterion polymer medium solution.

FIG. 22 shows the relationship between the zwitterion polymer (polyVimC3C) and an effect of a NaCl concentration in terms of a survival rate of K562 cells after cryopreservation.

FIG. 23 shows the relationship between the zwitterion polymer (polyVimC3C) and an effect of a NaCl concentration in terms of the survival rate of C6 cells after cryopreservation.

FIG. 24 shows the relationship between the zwitterion polymer (polyVimC3C) and a NaCl concentration in terms of the survival rate of OVMANA cells after cryopreservation.

FIG. 25 shows the relationship between the zwitterion polymer (polyVimC3C) and a NaCl concentration in terms of the proliferation rate of C6 cells after cryopreservation.

FIG. 26 shows the relationship between the zwitterion polymer (polyVimC3C) and a NaCl concentration in terms of the proliferation rate of OVMANA cells after cryopreservation.

FIG. 27 shows a micrograph showing the adhesive property of cells left to stand in the presence of the zwitterion polymer (polyVimC3C).

FIG. 28 shows a 1H-NMR chart of Poly(VimC₃C-co-C₁₆Vim).

FIG. 29 shows (a) the survival rate of BOSC cells after cryopreservation using Poly(VimC₃C-co-C₁₆Vim), (b) the survival rate of K562 cells after cryopreservation using Poly(VimC₃C-co-C₁₆Vim), and (c) the survival rate of OVMANA cells after cryopreservation using Poly(VimC₃C-co-C₁₆Vim).

FIG. 30 shows the toxicity of the zwitterion polymer Poly(VimC₃C-co-C₁₆Vim) to BOSC cells.

FIG. 31 shows the survival rate of K562 cells after cryopreservation using the zwitterion polymer Poly(VimC₃C-co-C₁₆Vim).

FIG. 32 shows results of adding Poly(VimC₃C-C16Vim) to cells, followed by observation by a confocal microscope.

FIG. 33 schematically shows the manner in which the zwitterion polymer of the present invention protects a cell membrane.

### DESCRIPTION OF EMBODIMENTS

In the present specification, a substance having a betaine structure is also referred to as an aprotic zwitterion or an aprotic zwitterion polymer, which is used in the same meaning as that of a zwitterion or a zwitterion polymer.

### (Biological Sample)

Examples of the biological sample to be treated with the preservative composition of the present invention include nucleic acids, proteins, organelles, cells, tissues, and individuals. Among them, organelles, cells, and tissues are preferred.

The origin of the cells to be used is not particularly limited. Examples thereof include animal cells, insect cells, plant cells, yeast cells, and bacterial cells. Examples of the animal cells include cells of human, mouse, rat, monkey, pig, dog, sheep, and goat. Examples of the bacterium include lactic acid bacteria, E. coli, Bacillus subtilis, and cyanobacteria.

Further, the type of the cells is not particularly limited and is, for example, appropriately selected from the group consisting of pluripotent stem cells, tissue stem cells, somatic cells, and germ cells. In this context, the “pluripotent stem cells” are a generic name for stem cells having the ability to differentiate into cells of every tissue (pluripotent differentiation). Examples thereof include embryonic stem cells (ES cells), induced pluripotent stem cells (iPS cells), embryonic germ stem cells (EG cells), and germ stem cells (GS cells). ES cells or iPS cells are preferred.

The “tissue stem cells” mean stem cells having the ability to differentiate into diverse cell species, though cell lineages into which the cells are capable of differentiating are limited to specific tissues (multipotent differentiation). Examples thereof include hematopoietic stem cells in the bone marrow, neural stem cells, hepatic stem cells, and skin stem cells.

The “somatic cells” refer to cells other than germ cells among cells constituting multicellular organisms. Preferred examples thereof include osteoclasts, fibroblasts, hepatic cells, pancreatic cells, muscle cells, bone cells, osteoblasts, cartilage cells, fat cells, skin cells, pancreatic cells, renal cells, lung cells, lymphocytes, erythrocytes, leucocytes, monocytes, and macrophages.

Examples of the “germ cells” include gametes for sexual generation, i.e., eggs, oocytes, sperms, and sperm cells, and spores for asexual generation.

The cells may be selected from the group consisting of sarcoma cells, established cell lines, and transformed cells. The “sarcoma” is a cancer which develops in connective tissue cells derived from non-epithelial cells of bone, cartilage, fat, muscle, blood, and the like, and includes, for example, soft tissue sarcoma and malignant bone tumor. The sarcoma cells are cells derived from sarcoma. The “established cell line” means cultured cells which have become capable of being maintained ex vivo over a long period, having constant stable properties, and being semipermanently subcultured. Examples thereof include PC12 cells (rat adrenal medulla-derived), CHO cells (Chinese hamster ovary-derived), HEK293 cells (human embryonic kidney-derived), HL-60 cells (human leucocyte-derived), and HeLa cells (human cervical adenocarcinoma-derived). The “transformed cells” mean cells with heritable nature altered by nucleic acid (e.g., DNA) transfer from the outside of cells. Animal cells, plant cells, or bacteria are transformed by use of a conventionally known method.

The culture of ES cells or iPS cells may involve, if necessary, feeder cells which are accessorily used for creating an environment necessary for cell proliferation or differentiation. Examples of the feeder cells include mouse fibroblasts. These feeder cells can be treated in advance by gamma ray irradiation or with an antibiotic so as not to proliferate.

### (Zwitterion Polymer)

In one aspect, the zwitterion polymer of the present invention is a zwitterion polymer of the following formula (1) or a labeled form thereof:

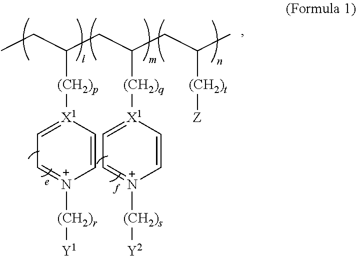

wherein
X¹ and X² are the same or different and each represent a carbon atom or a nitrogen atom;
Y¹ and Y² are the same or different and each represent an anion selected from the group consisting of —COO⁻, —SO₃⁻, —OP═O(H)O⁻, —OP═O(CH³)O⁻, and —OP═O(OR¹)O⁻;
Z represents a hydrogen atom, an optionally alkyl group-substituted aro hydrocarbon group having 6 to 10 carbon atoms, an optionally alkyl group-substituted 5- or 6-membered aromatic heterocyclic group, an optionally alkyl group-substituted nitrogen-containing heterocyclic ammonium salt, a tetraalkylammonium salt, a tetraphenylphosphonium salt, a tetraalkylphosphonium salt, a trialkylsulfonium salt, or a linear or branched alkyl group having 1 to 22 carbon atoms and optionally having 1 to 3 oxygen atoms in a molecular chain;
e and f each represent an integer of 0 or 1;
l, m, and n are each a number which indicates the content ratio of each repeat unit and represent a number which satisfies 0<l≤1, 0≤m<1, 0≤n<1, and l+m+n=1; and
p, q, r, s, and t each represent an integer of 0 to 6.

The zwitterion polymer of the present invention has an aprotic zwitterion, i.e., a cationic moiety and an anionic moiety, in a side chain of at least one repeat unit. The zwitterion structure is formed by a heterocyclic cation containing X¹ and a nitrogen atom (N) and an anion of Y¹ in the formula (1). The zwitterion polymer of the present invention may have a zwitterion structure formed by a heterocyclic cation containing X² and a nitrogen atom (N) and an anion of Y² in side chains of m repeat units.

In the present invention, the zwitterion structure is a generic name for compounds (intramolecular salts) which have a positive charge and a negative charge at non-adjacent positions in one molecule, have no dissociable hydrogen bonded to a positively charged atom, and have no charge as the whole molecule.

It is thus considered that the polymer of the present invention has a zwitterion in a side chain of a repeat unit, and thereby has a protective effect on cell membranes and a function as a cryopreservative. Furthermore, the polymer of the present invention shifts its state from a state associated through the side chain zwitterion into a dispersed state by the addition of one or more water-soluble compounds selected from the group consisting of an electrolyte, betaine, a zwitterion, an alcohol, a polyhydric alcohol, and a saccharide, and is therefore considered to act on cell membranes and exhibit a cryopreserving effect.

In the formula (1), l, m, and n are each a number which indicates the content ratio of each repeat unit and represent a number which satisfies 0<l≤1, 0≤m<1, 0<n<1, and l+m+n=1.

The formula (1) wherein m=n=0 represents a homopolymer having a functional group with a zwitterion structure in a side chain. The formula (1) wherein m represents a number exceeding 0 and n=0 represents a random or block copolymer having two types of zwitterion structures. The formula (1) wherein m=0 and n represents a number exceeding 0 represents a random or block copolymer having a repeat unit having a functional group with a zwitterion structure in a side chain, and a repeat unit having Z in a side chain. The formula (1) wherein m and n each represent a number exceeding 0 represents a ternary random or block copolymer.

p, q, and t each independently represent an integer of 0 to 6. Specifically, this moiety is a single bond or a linear alkylene group having 1 to 6 carbon atoms and is preferably a single bond, a methylene group, an ethylene group, a trimethylene group, or a tetramethylene group. This moiety is more preferably a single bond, a methylene group, or an ethylene group, further more preferably a single bond or a methylene group, from the viewpoint of cryoprotective effect on biological samples. p, q, and t are the same or different.

r and s each independently represent an integer of 0 to 6 and is preferably an integer of 1 to 6. This moiety is preferably a linear alkylene group having 1 to 6 carbon atoms. Specifically, this moiety is preferably a methylene group, an ethylene group, a trimethylene group, a tetramethylene group, a pentamethylene group, or a hexamethylene group. r and s are the same or different.

In the formula (1), X¹ and X² are the same or different and each represent a carbon atom or a nitrogen atom.

In the formula (1), examples of the heterocyclic cation containing X¹ and a nitrogen atom (N) include an imidazolium cation (X¹═N, e=0), a pyridinium cation (X¹═C, e=1), a pyrrolinium cation (X¹═C, e=0), and a pyrazinium cation (X¹═N, e=1). Among them, an imidazolium cation (X¹═N, e=0) or a pyridinium cation (X¹═C, e=1) is preferred.

In the formula (1), examples of the heterocyclic cation containing X² and a nitrogen atom (N) include an imidazolium cation (X²═N, f=0), a pyridinium cation (X²═C, f=1), a pyrrolinium cation (X²═C, f=0), and a pyrazinium cation (X²═N, f=1). Among them, an imidazolium cation (X²═N, f=0) or a pyridinium cation (X²═C, f=1) is preferred.

Y¹ and Y² are the same or different and each represent an anion selected from the group consisting of —COO⁻, —SO₃⁻, —OP═O(H)O⁻, —OP═O(CH₃)O⁻, and —OP═O(OR¹)O⁻. Each of Y¹ and Y² is preferably an anion selected from the group consisting of —COO⁻, —SO₃⁻, and —OP═O(OH)O⁻, more preferably an anion selected from the group consisting of —COO⁻ and —SO₃⁻, further more preferably —COO⁻.

Z represents a hydrogen atom, an optionally alkyl group-substituted aromatic hydrocarbon group having 6 to 10 carbon atoms, an optionally alkyl group-substituted 5- or 6-membered aromatic heterocyclic group, an optionally alkyl group-substituted nitrogen-containing heterocyclic ammonium salt, a tetraalkylammonium salt, a tetraphenylphosphonium salt, a tetraalkylphosphonium salt, a trialkylsulfonium salt, or a linear or branched alkyl group having 1 to 22 carbon atoms and optionally having 1 to 3 oxygen atoms in a molecular chain.

Specific examples thereof include a hydrogen atom, a phenyl group, a naphthyl group, a C1-C22 alkyl-substituted phenyl group, a C1-C22 alkyl-substituted naphthyl group, an imidazolyl group, a triazolyl group, a pyridyl group, a pyrrolyl group, a pyrazinyl group, a furyl group, a thienyl group, an oxazolyl group, a thiazolyl group, pyridinium chloride, a C1-C22 alkylpyridinium chloride, imidazolinium chloride, a C1-C22 alkylimidazolinium chloride, pyridinium bromide, a C1-C22 alkylpyridinium bromide, imidazolinium bromide, a C1-C22 alkylimidazolinium bromide, pyrrolidinium chloride, a C1-C22 alkylpyrrolidinium chloride, pyrrolidinium bromide, a C1-C22 alkylpyrrolidinium bromide, tetramethylammonium chloride, a C2-C22 alkyltrimethylammonium chloride, tetramethylammonium bromide, a C2-C22 alkyltrimethylammonium bromide, tetraphenylphosphonium chloride, tetraphenylphosphonium bromide, trimethylsulfonium chloride, trimethylsulfonium bromide, a C1-C22 linear or ranched alkyl group, an ethoxyethyl group, an ethoxyethoxyethyl group, and an ethoxyethoxyethoxyethyl group.

Preferred examples of Z include an optionally alkyl group-substituted 5- or 6-membered aromatic heterocyclic group and an optionally alkyl group-substituted nitrogen-containing heterocyclic ammonium salt, more preferably an imidazolyl group, a triazolyl group, a pyridyl group, a pyrrolyl group, a pyrazinyl group, a furyl group, a thienyl group, an oxazolyl group, a thiazolyl group, pyridinium chloride, a C1-C22 alkylpyridinium chloride, imidazolinium chloride, a C1-C22 alkylimidazolinium chloride, pyridinium bromide, a C1-C22 alkylpyridinium bromide, imidazolinium bromide, a C1-C22 alkylimidazolinium bromide, pyrrolidinium chloride, a C1-C22 alkylpyrrolidinium chloride, pyrrolidinium bromide, and a C1-C22 alkylpyrrolidinium bromide,
further more preferably an imidazolyl group, a pyridyl group, pyridinium chloride, a C1-C22 alkylpyridinium chloride, imidazolinium chloride, a C1-C22 alkylimidazolinium chloride, pyridinium bromide, a C1-C22 alkylpyridinium bromide, imidazolinium bromide, and a C1-C22 alkylimidazolinium bromide.

Examples of the labeled form of the zwitterion polymer of the formula (1) include radioisotope labels, enzyme labels, chemiluminescent material labels, and fluorescent material labels. Among them, a chemiluminescent material label or a fluorescent material label is preferred. Examples of the fluorescent labeling material include fluorescein, cyanin, BODIPY, dansyl, pyranine, coumarin, carbopyronine, and phycocyanin.

Examples of the labeling of the zwitterion polymer using such a labeling material include an approach of bonding the labeling material to a side chain of the zwitterion polymer, and an approach of copolymerizing a monomer with the labeling material bonded to a side chain. For example, one or more monomers constituting the polymer of the formula (1) can be copolymerized with fluorescein acrylate to obtain a fluorescein-labeled form.

The molecular weight of the polymer compound used in the present invention needs to be taken into consideration in terms of a backbone structure, a side chain structure, a ratio to the whole, and the like. A measurement approach known in the art can be employed which reflects each feature. The zwitterion polymer is known to vary in backbone conformation depending on the type of a zwitterion and depending on pH or an electrolyte (e.g., NaCl) concentration.

A possible backbone structure of the zwitterion polymer of the present invention is, for example, a star-shaped, comb-shaped, or cross-linked structure, and any structure may be used for the objects of the present invention, however, the backbone conformation varies depending on the type of a side chain. In the case of a polymer having a linear backbone, its dispersibility in water can be enhanced by expanding the polymer by the addition of, for example, an electrolyte such as NaCl. The zwitterion polymer of the present invention preferably has a large molecular weight to a certain degree from the viewpoint of a cell membrane structure or cell permeability and because of issues related to extracellular formation of ice crystals and solubility of a poorly soluble substance, depending on a zwitterion structure present in a side chain. In the state of the molecular chain expanded by an electrolyte, on the polyethylene oxide basis, Mw is preferably 10000 or larger, and Mn is preferably 5000 or larger, Mw is more preferably 10000 or larger and 2000000 or smaller, and Mn is more preferably 5000 or larger and 1500000 or smaller.

Further, the molecular weight distribution of the polymer is also taken into consideration. In the case of using the zwitterion polymer as the cryopreservative for biological samples, it is preferred that the zwitterion polymer of the present invention does not penetrate cell membranes, because extracellular formation of ice crystals is promoted.

The zwitterion polymer of the present invention is excellent in cryopreserving effect. The polymer of the present invention enhances a cryopreserving effect through mechanisms different from those of DMSO and the like conventionally used in cryopreservation. As a possible mechanism, DMSO is considered to penetrate cells, whereas the zwitterion polymer of the present invention, which does not infiltrate into cells, forms a matrix outside cells and accumulates outside the cells and in the vicinity of cell membranes. A reason for this is presumably that a zwitterion molecule has a betaine structure and therefore has a charge so that the zwitterion polymer cannot infiltrate into cell membranes. Furthermore, a polymer side chain has a hydrophobic functional group having high affinity for cell membranes. When the functional group is inserted into cell membranes from outside cells, it is considered that the hydrophilic polymer is anchored to the cell membranes outside the cells so that the polymer accumulates outside the cells and in the vicinity of the cell membranes. Examples of the functional group which contributes to such accumulation include an alkylene group positioned between the cation and the anion in the formula (1). Further, Z preferably has a C1-C22 alkyl group, more preferably has a C3-C18 alkyl group. The abundance ratio of such a substituent is preferably 0.001 to 10 mol % or 0.01 to 1 mol %, more preferably 0.1 to 0.5 mol %, to the whole polymer.

### (Method for Producing Zwitterion Polymer)

The zwitterion polymer of the present invention can be produced by polymerization such as radical polymerization, anionic polymerization, or cationic polymerization using an α-olefin monomer corresponding to the repeat unit. These polymerization methods may be appropriately employed differently depending on the characteristics of the monomer, the purpose or properties of the polymer to be produced, and the like. In the case of using only one type of α-olefin having a betaine structure in polymerization reaction, a homopolymer of zwitterion polymers having the same side chain (in the formula (1), l=1, m=n=0, or m=1, l=n=0) can be produced. In the case of using two types of α-olefins having different betaine structures in polymerization reaction, a copolymer of zwitterion polymers having two types of side chains having the different betaine structures (0<l<1, 0<m<1, n=0) can be produced. In the case of using one type of α-olefin having a betaine structure and general α-olefin in the polymerization reaction, a zwitterion polymer of polyolefin having the betaine structure in a side chain (l=0, 0<m<1, 0<n<1, or 0<l<1, m=0, 0<n<1) can be produced. In the production of the zwitterion polymer of polyolefin having the betaine structure in a side chain (l=0, 0<m<1, 0<n<1, or 0<l<1, m=0, 0<n<1), this polymer may be produced by a method of introducing an anion group in Y¹ and Y² to a nitrogen atom in a side chain of a polymer having a structure of the formula (1) without (CH₂)rY¹ and (CH₂)sY², and cationizing the nitrogen atom to construct a betaine structure in the side chain (this method is also referred to as “post-modification”). A polymer known in the art may be used as the polymer having a structure of the formula (1) having neither (CH₂)rY¹ nor (CH₂)sY². If such a polymer is unknown, the polymer having a structure of the formula (1) having neither (CH₂)rY¹ nor (CH₂)sY² may be produced by polymerization.

The radical polymerization for the zwitterion polymer of the present invention may be performed by a high-temperature and high-pressure production method. Alternatively, the production can be performed at ordinary pressure as long as a suitable polymerization initiator is used. Examples of the polymerization initiator which can be used include azo compounds such as 2,2′-azobisisobutyronitrile, and peroxides such as benzoyl peroxide. The polymerization initiator is used in the range of the proportion of 0.001 mol % or more, preferably 0.01 mol % or more, more preferably 0.1 mol % or more, and 10 mol % or less, preferably 5 mol % or less, more preferably 1 mol % or less, to the α-olefin having a betaine structure. The molecular weight of the polymer to be produced can be adjusted by the amount of the polymerization initiator used as well as properties or the presence or absence of a solvent used, a temperature, a pressure, and the like.

Examples of the method for cationizing a nitrogen atom and introducing an anion group constituting the betaine structure include a method of reacting a polymer having a nitrogen atom in a side chain moiety with halogenated fatty acid ester or alkylsultone. Examples of the polymer having a nitrogen atom in a side chain moiety include polypyridine and polyimidazoline.

### (Adjustment of Osmotic Pressure)

As shown in Examples described later, an aqueous solution of the zwitterion polymer of the present invention may have a low osmotic pressure due to its large molecular weight, though the zwitterion polymer is water-soluble. However, when a compound which contributes to an osmotic pressure is added to the aqueous solution of the zwitterion polymer of the present invention, effects of improving the osmotic pressure and remarkedly improving the survival rate of biological samples after cryopreservation are obtained.

In the case of using the zwitterion polymer of the present invention in a dispersion containing a biological sample such as cells, one or more water-soluble compounds selected from the group consisting of an electrolyte, betaine, a zwitterion, an alcohol, a polyhydric alcohol, and a saccharide are preferably added to the dispersion in order to adjust the osmotic pressure. The osmotic pressure adjustment can further improve a maintaining effect on the survival and functions of the biological sample when the biological sample is cryopreserved. Thus, a composition comprising the zwitterion polymer of the present invention is useful as a preservative composition for biological samples, particularly, as a cryopreservative composition for biological samples. The form of the preservative composition for biological samples of the present invention may be a medium composition for biological samples. Among them, an electrolyte is more preferred for the osmotic pressure adjustment from the viewpoint of a cryopreserving effect on biological samples. The electrolyte is preferably sodium chloride, potassium chloride, sodium phosphate, potassium phosphate, sodium bicarbonate, or potassium bicarbonate in consideration of toxicity to biological samples.

When one or more water-soluble compounds selected from the group consisting of an electrolyte, betaine, a zwitterion, an alcohol, a polyhydric alcohol, and a saccharide is added to an aqueous solution, a medium, a cryopreservative solution or the like containing the zwitterion polymer of the present invention, a more significant effect is obtained when used. These water-soluble compounds act on a zwitterion side chain of the zwitterion polymer to change the conformation of the entire polymer. It is considered that if a water-soluble compound such as an electrolyte, which acts on a side chain of the zwitterion polymer is incorporated into the zwitterion polymer, for example by forming a salt with the side chain, a concentration which contributes to elevation in osmotic pressure cannot be obtained. Therefore, the type and amount of the water-soluble compound to be added must be taken into consideration in relation to the structure of the zwitterion polymer.

Here, the electrolyte is a substance to be ionized into a cation and an anion when dissolved in a solvent.

Preferred examples of the cation include sodium ions, potassium ions, calcium ions, and magnesium ions. Examples of the anion include chloride ions, phosphate ions, and hydrogen carbonate ions. Thus, the electrolyte is preferably a compound having a combination of a cation selected from the group consisting of a sodium ion, a potassium ion, a calcium ion, and a magnesium ion, and an anion selected from the group consisting of a chloride ion, a phosphate ion, and a hydrogen carbonate ion. Specific examples thereof include sodium chloride, potassium chloride, sodium phosphate, potassium phosphate, sodium bicarbonate, and potassium bicarbonate.

Examples of the alcohol include alcohols having 1 to 6 carbon atoms, such as methanol, ethanol, and isopropanol. Examples of the polyhydric alcohol include water-soluble polyhydric alcohols such as ethylene glycol and propylene glycol. Examples of the saccharide include monosaccharides and disaccharides, such as glucose and sucrose. Examples of the betaine include carnitine. Examples of the zwitterion include zwitterions described in WO2020/230721.

### (Aggregability of Polymer)

It was observed from the particle size measurement by dynamic light scattering (DLS) that in the aqueous solution of the zwitterion polymer of the present invention, many particles were present at from 100 to 1000 nm. Further, it was observed that when an electrolyte was added thereto, the degree of aggregation thereof was weakened, and the aggregation was rare in some samples. After electrolyte addition, the presence of many particles was observed at or around 10 nm (FIG. 19).

The zwitterion polymer of the present invention is aggregated, depending on the density of side chains, in an aqueous solution. The aggregated polymer forms an ion pair with an electrolyte through the interaction between the electrolyte and the side chains so that the shape of the aggregated polymer is expanded by conformational change of the polymer backbone, resulting in a dispersed state. Although the addition of the electrolyte elevates an osmotic pressure, the zwitterion side chains as zwitterion monomers are also considered to be converted into a more dispersed state and contribute to preservability.

As seen from observation by DLS, the zwitterion polymer of the present invention contributes to preservability through the more dispersed state of the aggregated polymer in the aqueous solution containing the electrolyte. The dispersed state of the polymer allows the polymer to be widely dispersed so as to cover biosurface with the polymer backbone. Thus, the side chains are considered to interact with biomembranes or enter cell membranes, thereby enhancing affinity for the biomembranes. As a result, the zwitterion polymer interacts with the biological sample to be preserved, particularly, cell membranes, to form matrix-like matter in the vicinity of cells. Such interaction between the zwitterion polymer and cell membranes also influences the relationship between cells, and an aggregated state of cells is observed (FIG. 27). Among cells observed in a medium containing the zwitterion polymer of the present invention added, some cells are observed to gather. This manner stands in contrast to cells in PBS where the cells are observed in isolation from each other. Thus, the addition of the zwitterion polymer of the present invention can be expected to cause some effect and exert an adhesive effect between cells. Although the degree of the adhesive effect is uncertain, it is expected that such an effect will be useful for organ engraftment by surgery such as organ transplantation or for tissue or organ production using a three-dimensional printer, for example.

An elevating effect on an ice melting temperature was observed by DSC measurement of a temperature at which ice was melted in a temperature increase step of bringing the temperature back to room temperature after rapid freezing. As seen from the obtained results, the dispersed state of the zwitterion polymer ascribable to the electrolyte is expected to cause some structural change through interaction with cell membranes and exert a cell protective effect. For example, such a protective effect is considered to suppress the influx of ice into cells and maintain the survival of the cells. Furthermore, an extracellular protective effect of the zwitterion polymer also enables animal cells to be cultured as floating cells independently one by one in a medium.

### (Preservative Composition)

The preservative composition for biological materials of the present invention has a function as the cryopreservative for the biological materials and also has a function as a composition for maintening the preservation of the biological materials. Specific examples thereof include cryopreservative compositions for biological materials, compositions for culturing biological materials, medium compositions for biological materials, compositions for preserving biological materials, compositions for functional maintenance of biological materials, and composition for functional test of biological materials.

Examples of the vehicle in the preservative composition for biological samples of the present invention include water and mixtures of water and organic solvents. The selected vehicle is preferably a substance which ionizes an electrolyte into a cation and an anion. In this context, examples of the organic solvent include lower alcohols such as methanol and ethanol, dimethyl sulfoxide (DMSO), and dimethylformamide (DMF).

The concentration of the zwitterion polymer when the zwitterion polymer of the present invention or the labeled form thereof is used as the preservative for biological samples is not particularly limited and is preferably 2 mass % or more and 30 mass % or less, more preferably 5 mass % or more and 30 mass % or less, further more preferably 10 mass % or more and 20 mass % or less.

The concentration of one or more water-soluble compounds selected from the group consisting of an electrolyte, betaine, a zwitterion, an alcohol, a polyhydric alcohol, and a saccharide is preferably a concentration having no influence on the survival of biological samples, and is preferably 0.2 mass % or more and 5 mass or less, more preferably 0.5 mass or more and 3 mass % or less, further more preferably 0.5 mass % or more and 2 mass % or less.

In the preservative composition of the present invention, a cell-penetrating substance may be additionally used as an additive per 100 parts by weight of a composition comprising the aprotic zwitterion and water or a composition consisting of the aprotic zwitterion and water (also referred to as an aqueous aprotic zwitterion solution). The cell-penetrating substance can be added, for use, at usually at least 1 part by weight, preferably 10 parts by weight or more, per 100 parts by weight of the aqueous aprotic zwitterion solution, and the upper limit thereof is usually 30 parts by weight or less, preferably 20 parts by weight or less, more preferably 15 parts by weight or less. In the present specification, 1 part by weight added per 100 parts by weight of the aqueous solution of the aprotic zwitterion is also referred to as 1% based on the aqueous solution of the aprotic zwitterion. Examples of such a cell-penetrating substance include dimethyl sulfoxide (DMSO), glycerol, ethylene glycol, and propylene glycol. Such a cell-penetrating substance can further improve cryopreservability.

The amount of such a cell-penetrating substance to be added may be reduced or increased from an amount usually used. For example, the amount of DMSO to be added may be 3 mass % or more and 25 mass % or less. The amount of glycerol to be added may be 3 mass % or more and 25 mass % or less.

The preservative composition of the present invention may contain a medium component for biological sample culture. In this case, the preservative composition of the present invention has a function as a medium composition or a composition for culture. Examples of the medium component for biological sample culture include medium components for cell cultures and specifically include inorganic salts, buffers, carbohydrates, vitamins, proteins, peptides, fatty acids, lipids, trace elements, serum, hormones, growth factors, signaling substances, and antibiotics. The composition of the present invention, when used as a medium composition, preferably contains such a medium component.

The preservative composition of the present invention improves the cryopreservability of cells or the like in a dispersion and is therefore also useful as an additive for media for cells or the like. Thus, the composition of the present invention comprising the preservative and a medium component for cells can be used as a medium for cryopreservation of cells or the like. In this context, examples of the medium component for cells include nutrients (e.g., saccharides for cell proliferation), and peptides and proteins (e.g., serum, and proteins and peptides purified from serum), in addition to the zwitterion polymer of the present invention, the electrolyte, and the cell-penetrating substance.

In a case where the preservative composition of the present invention is used when a biological sample (e.g. cells) dispersion is frozen by a slow freezing or rapid freezing method and then thawed, the survival rate of cells can be improved. Here, cell freezing conditions in the slow freezing method can be appropriately set in accordance with conventional conditions. Specifically, the cells can be cooled to 0 to −200° C. at a cooling rate of, for example, −0.1 to −15° C./min. The rapid freezing method is preferably performed, for example, using a cooling rate of −15 to −20000° C./min and a cooling temperature in the range of 0 to −200° C.

As a method of thawing the cryopreserved cells, for example, it is preferred to rapidly transfer an ampule containing the frozen cells to a water bath of 37° C. for thawing. The contents of the ampule are transferred to a sterile tube using a pipette. Then, a preheated medium already supplemented with a suitable supplement is gradually added thereto. A live cell density is measured using trypan blue. A suitable amount of the cell suspension is transferred to a flask and inoculated at a cell density recommended by a cell line data sheet.

### (Dissolution of Poorly Soluble Substance)

As used herein, the “poorly soluble substance” refers to a substance having a property of being dissolved only slightly in water, and is a substance having solubility (25° C.) of 1% by weight or less, preferably 0.5% by weight or less, particularly preferably 0.1% by weight or less, in water. Such a poorly soluble substance is a medicament, a medicament for an animal, a quasi-drug, a cosmetic, a substance serving as an active ingredient in agricultural chemicals (also including a candidate substance capable of serving as an active ingredient), a food additive, an organism-derived substance, or a plant-derived substance, and includes low-molecular substances, and oligomer and polymer substances such as oligopeptides, polypeptides, polysaccharides, DNA, and RNA.

The “poorly soluble medicament” corresponds to “sparingly soluble”, “slightly soluble”, “very slightly soluble”, and “practically insoluble” drugs defined in the Japanese Pharmacopoeia. Specific examples thereof include antitumor agents, antibiotics, antihyperlipidemic agents, antibacterial agents, therapeutic agents for allergic disease, antihypertensive agents, therapeutic agents for arteriosclerosis, blood circulation promoting agents, hormonal agents, fat-soluble vitamins, antidiabetic agents, antiandrogen agents, cardiotonic drugs, drugs for arrhythmias, anti-inflammatory agents, sedative hypnotics, tranquilizers, antiepileptic agents, antidepressants, therapeutic agents for gastrointestinal agent disease, drugs for diuresis, local anesthetics, anticoagulants, antihistamic agents, antimuscarinic agents, antimycobacterial agents, immunosuppressants, antithyroid agents, antiviral agents, anxiolytic agents, astringents, β-adrenoreceptor blockers, agents exerting inotropic action on cardiac muscle, contrast media, corticosteroids, cough suppressing agents, diagnostic agents, diagnostic imaging agents, diuretics, dopamine agonists, lipid adjusters, muscle relaxers, parasympathetic drugs, thyrocalcitonin, prostaglandin, radioactive medicaments, sex hormones, stimulants, appetite suppressing agents, sympathetic agents, thyroid drugs, vasodilators, isoflavone, and xanthene.

More specific examples of the poorly soluble substance include glycyrrhetinic acid and its salts, glycyrrhizic acid and its salts, coumarin, ononin, liquiritin, peptides, polypeptides such as collagen, polysaccharides such as xylan, lignin, and chloramphenicol.

The addition of the zwitterion polymer of the present invention can improve the solubility of such a poorly soluble substance. Further, when the poorly soluble substance is dissolved in a solution containing the zwitterion polymer of the present invention and one or more water-soluble compounds selected from the group consisting of an electrolyte, betaine, a zwitterion, an alcohol, a polyhydric alcohol, and a saccharide, the solubility of the poorly soluble substance is remarkably improved.

### EXAMPLES

Next, the present invention will be described in more detail with reference to Examples. However, the present invention is by no means limited to these Examples.

### (Pure Water)

In Examples, water purified by an ultrapure water production apparatus manufactured by Sartorius AG was used.

### (Cell)

The mouse fibroblasts (mNF) (mouse normal fibroblast) established from C57BL/6-EGFP mice were used. Human kidney cells (BOSC) and rat glioma cells (C6 cells) were obtained from Division of Tumor Cell Biology and Bioimaging Cancer Research Institute of Kanazawa University. Human chronic myeloid leukemia cells (K562 cells) and mouse neuroglial and neuronal character coexpressing cells (Vn1919 cells) were obtained from Richard Wong Laboratory at Kanazawa University. OVMANA cells were purchased from JCRB Cell Bank of National Institutes of Biomedical Innovation, Health and Nutrition.

### (¹H-NMR)

¹H-NMR was measured by ECA400 manufactured by JEOL Ltd. (external magnetic field at 400 MHz).

### (GPC)

As the stationary phase, TSKgel α-M column (Tosoh Corp.) was used. As the mobile phase, pure water was used. The flow rate was set to 1.0 mL/min, and the measurement temperature was set to 40° C. Detection was carried out by a refractive index detector (RID-10A). Various molecular weights were determined on a polyethylene oxide basis.

### (DSC)

A DSC apparatus manufactured by Shimadzu Corp. was used.

The temperature was lowered to −150° C. at 1° C./min and then brought back to room temperature at 5° C./min in a temperature increase step to measure the temperature at which ice melted.

### (DLS)

The particle size distribution was determined by a DLS apparatus manufactured by Horiba, Ltd.

A solution passed through a 3 μm filter was placed in a disposable cell, and the particle size was measured at a set temperature of 25° C.

### (Osmotic Pressure)

A vapor pressure-type osmometer (VAPRO 5600; Wescor, Inc., Logan, UT, USA) was used, and an osmotic pressure was measured in a 10 mL standard chamber (room temperature: 25° C., apparatus sensor temperature: 33° C., sample volume: 10 μL).

### (Cryopreservation Test)

### (a) Cryopreservation of Cell

In the case of adherent cells, the cells to be frozen were treated with trypsin and recovered by centrifugation. In the case of floating cells, the cells to be frozen were recovered by centrifugation. The cells of each type were diluted with Dulbecco's modified Eagle medium (DMEM), and the cell concentration was measured. In the present specification, the DMEM solution is also simply referred to as a medium.

Subsequently, each dilution was dispensed at 1 mL/tube to 1.5 mL tubes, and the cells were suspended in a cryopreservative solution containing 100 μL of a zwitterion polymer solution and frozen at a cooling temperature of −85° C. and a cooling rate of −1° C./min by a cell freezing container Mr. Frosty®. In the present Examples, the commercially available cryopreservative solution used was CultureSure® (manufactured by FUJIFILM Wako Pure Chemical Corp.) unless otherwise specified. The cryopreservation time was set to 48 hours. Then, the cells were thawed at a tube temperature of 37° C. and used in live cell counting.

### (b) Thawing of Cell and Live Cell Counting

The cells were thawed by the addition of 1 mL of a medium to the cryopreservation vial at 37° C. and centrifuged to remove a supernatant. The cells thus obtained by centrifugation were resuspended in a medium, followed by live cell counting.

### (Cytotoxicity Test)

Cells were left to stand in a polymer solution at room temperature for 60 minutes. Then, the proportion of dead cells in the cells was measured by trypan blue staining.

### (Cell Proliferation Test)

Cells were cryopreserved in accordance with the cryopreservation test described above, and then cultured for a given period in a medium (DMEM containing 10% FBS and 1% antibiotic), and the number of cells which proliferated from before culture was measured.

### Example 1

### (1) Production of VimC₃C

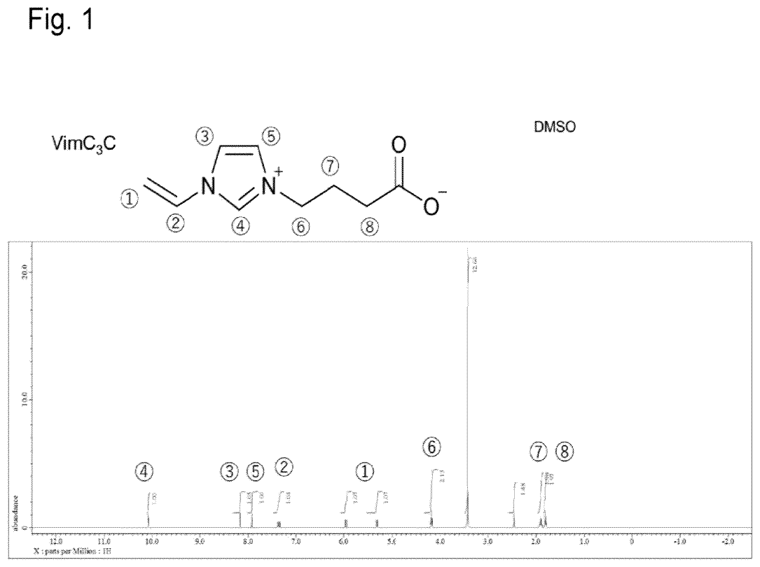

To 100 mb of tetrahydrofuran (FUJIFILM Wako Pure Chemical Corp.), 15.69 g (0,167 mmol) of vinylimidazole (Tokyo Chemical Industry Co., Ltd.) was added, then 32.48 g (0,167 mmol) of 4-bromobutyric acid ethyl ester (Tokyo Chemical Industry Co., Ltd.) was added, and the mixture was refluxed at 70° C. for 24 hours. The reaction mixture was washed with ethyl acetate and then applied to an anion exchange resin (Amberlite IRN 78A) column. The solvent in the eluate was distilled off under reduced pressure to obtain VimC₃C. FIG. 1 shows a ¹H-NMR chart of VimC₃C (DMSO).

### (2) Production of Poly(VinC₃C)

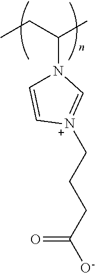

To 10 mL of pure water, 3 g (0.014 mmol) of VimC₃C was added, then 22.83 g (0.0014 mmol) of 2,2′-azobis(isobutyronitrile) (Tokyo Chemical Industry Co., Ltd.) was added as a polymerization initiator, and the mixture was stirred at 80° C. for 16 hours. The reaction mixture was dialyzed with pure water and dried under reduced pressure to obtain Poly(VimC₃C).

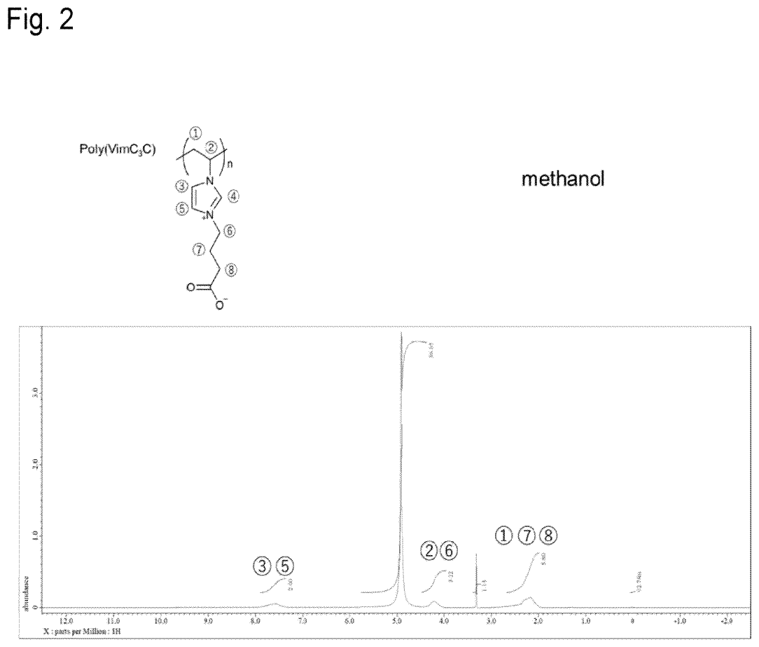

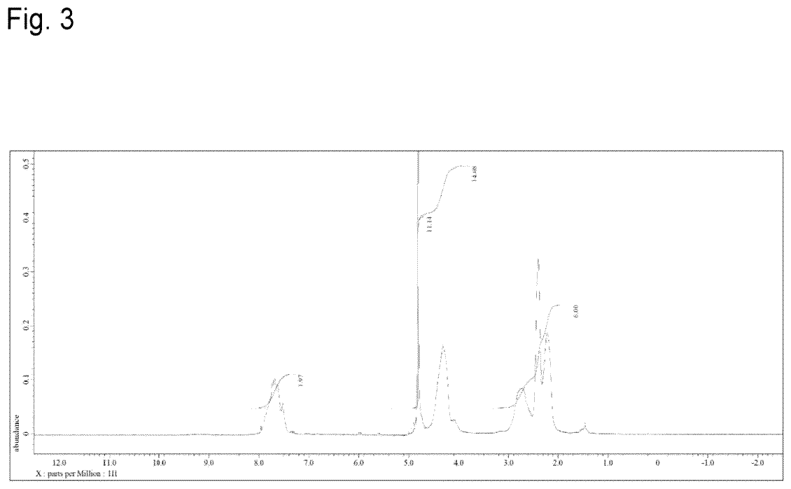

FIG. 2 shows ¹H-NMR of Poly(VimC₃C) (methanol). FIG. 3 shows 1H-NMR of a solution obtained by adding NaCl to a D₂O solution of the polymer.

The reaction was carried out by adding a polymerization initiator in an amount corresponding to the monomer ratio of 0.01 to 10 mol %. In Examples of the present specification, a solution in which a polymer produced by the addition of the polymerization initiator at a monomer ratio of 10 mol % was added at 10 mass based on the whole solution, as in this Example, is referred to as 10% 10 mol % Poly(VimC₃C). The polymer produced by the addition of the polymerization initiator at the monomer ratio of 1 mol % was used, unless the amount of the polymerization initiator is otherwise specified.

### Example 2

### Method for Producing Poly(VpyC₃C)

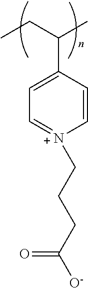

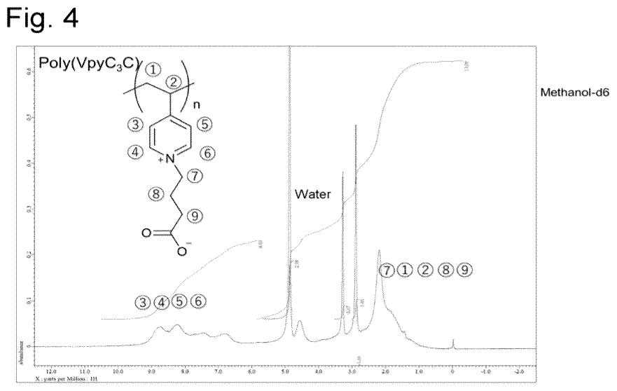

To 20 mb of dimethyl sulfoxide (Kanto Chemical Co., Inc.), 4.95 g (0.0033 mmol) of poly(4-vinylpyridine) (Merck Japan K.K.) was added, then 3.2 g (0.0033 mmol) of 4-bromobutyric acid ethyl ester (Tokyo Chemical Industry Co., Ltd.) was added, and the mixture was stirred at room temperature for 24 hours. The reaction mixture was washed with ethyl acetate and dried under reduced pressure to obtain Poly(VpyC₃C), FIG. 4 shows ¹H-NMR chart of Poly(VpyC₃C) (methanol-d6) thus obtained. Mw=34321, Mn=19981, melting point (Tm)=149° C.

### Example 3

### Method for Producing Poly(VpyC₃S)

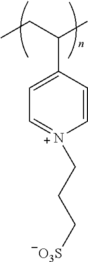

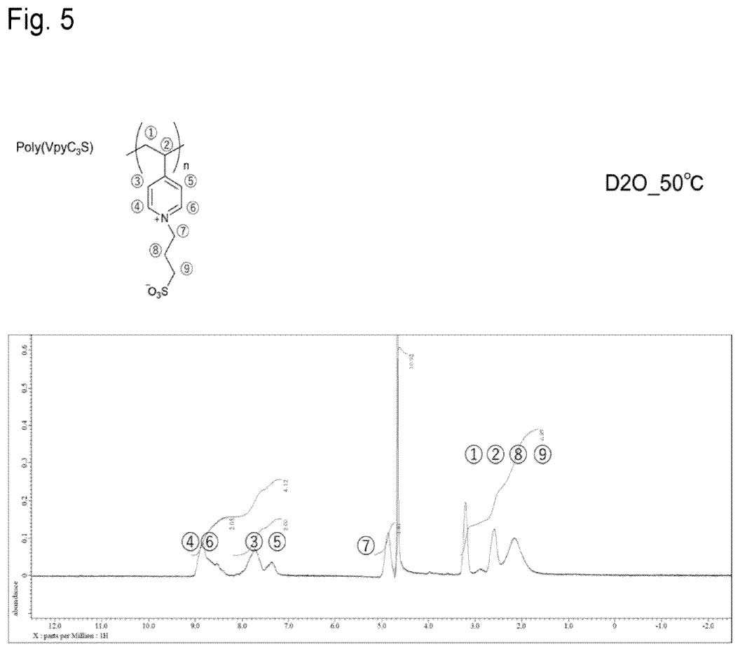

To 10 mL of dichloromethane (Kanto Chemical Co., Inc.), 0.92 g (0.0018 mmol) of poly(4-vinylpyridine) (Merck Japan K.K.) was added, then 1.1 g (0.0018 mmol) of 1,3-propanesultone (Tokyo Chemical Industry Co., Ltd.) was added, and the mixture was stirred at room temperature for 24 hours. The reaction mixture was washed with ethyl acetate and dried under reduced pressure to obtain Poly(VpyC₃S). FIG. 5 shows a 1H-NMR chart of Poly(VpyC₃S) (D₂O, 50° C.) thus obtained.

### Example 4

### (1) Production of VimC₃S

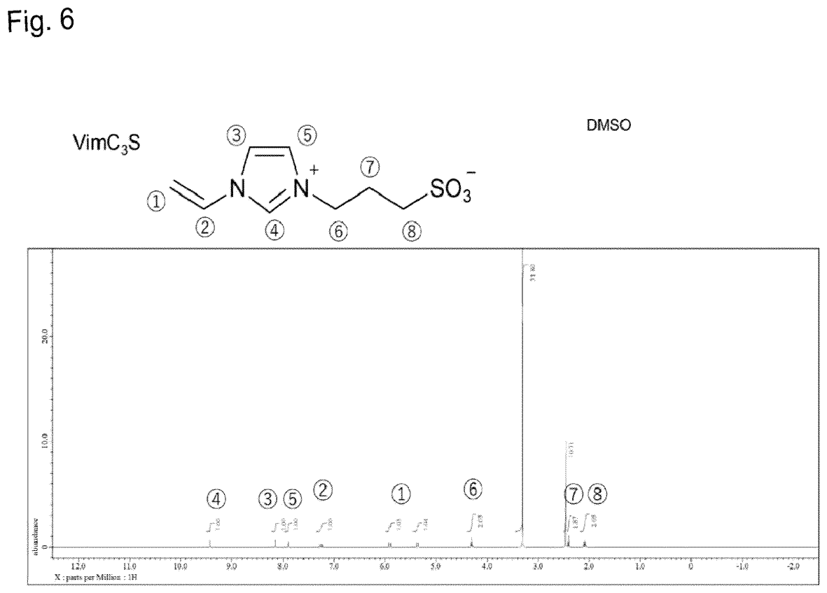

To 150 mL of tetrahydrofuran (FUJIFILM Wako Pure Chemical Corp.), 21.76 g (0.23 mmol) of vinylimidazole (Tokyo Chemical Industry Co., Ltd.) was added, then 28.24 g (0.23 mmol) of 1,3-propanesultone (Tokyo Chemical Industry Co., Ltd.) was added, and the mixture was refluxed at room temperature for 24 hours. The reaction mixture was washed with ethyl acetate and then dried under reduced pressure to obtain VimC₃S. FIG. 6 shows a 1H-NMR chart of VimC₃S (D₂O) thus obtained.

### (2) Production of Poly(VimC₃S)

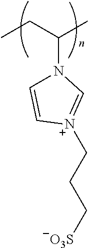

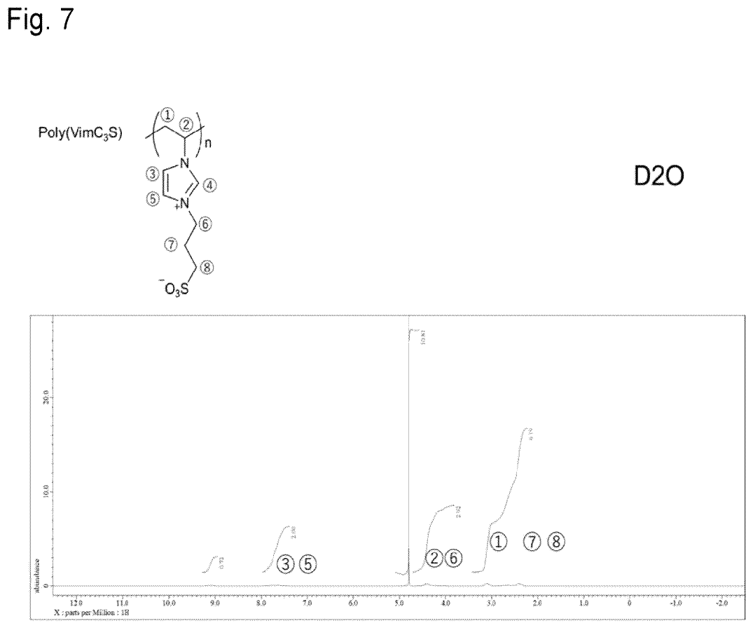

To 10 mL of a 2% aqueous NaCl solution (Nacalai Tesque, Inc.), 3 g (0.017 mmol) of VimC₃S was added, then 27.42 g (0.00017 mmol) of 2,2′-azobis(isobutyronitrile) (Tokyo Chemical Industry Co., Ltd.) was added, and the mixture was stirred at 80° C. for 16 hours. The reaction mixture was reprecipitated with pure water and dried under reduced pressure to obtain Poly(VimC₃S). FIG. 7 shows a 1H-NMR chart of Poly(VimC₃S) (DO) thus obtained.

### Example 5-1

### Production of Poly(VpyC₃C)50

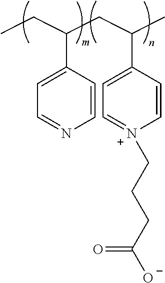

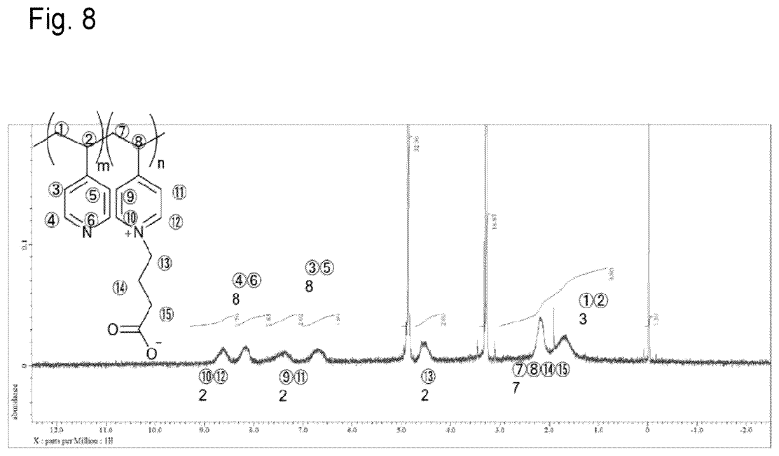

Poly(4-vinylpyridine) (Mw: 160000, Merck Japan K.K.) (5.53 g, 0.01 mmol) was dissolved in dichloromethane. To the solution, 4-bromobutyric acid ethyl ester (2.31 g, 0.01 mmol, Tokyo Chemical Industry Co., Ltd.) was added, and the mixture was refluxed at 50° C. for 17 hours. The reaction mixture thus refluxed was washed with ethyl acetate, purified with an ion exchange resin (name: Amberlite IRN78 hydroyide dsm), and then dialyzed using Spwctra/por 7 Dialysis Membrane Pre-treated RC Tubing MWCO 1 kD to obtain 4.88 g of Poly(VpyC₃C)50. FIG. 8 shows a ¹H-NMR (CH₃OH-d) chart. m:n=1:1 from 1H-NMR signals. Mw=249086, Mn=204914.

### (Example 5-2) Poly(VpyC₃C)40

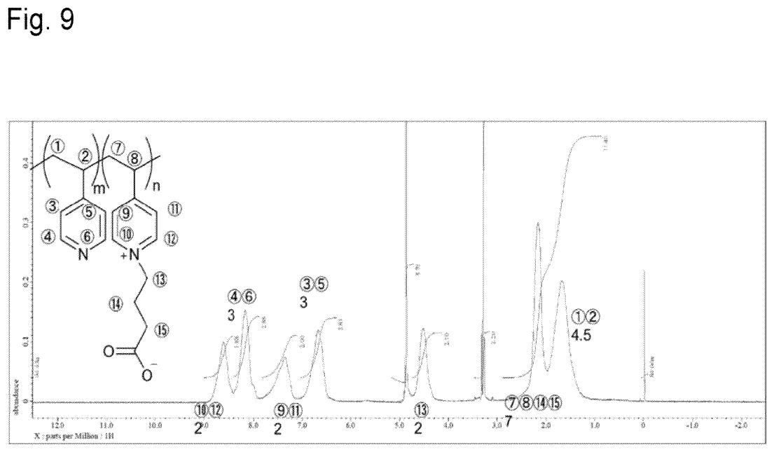

3.59 g of Poly(VpyC₃C)40 was obtained by the same operation as in Example 5-1 except that 4-bromobutyric acid ethyl ester (1.91 g, 0.008 mmol, Tokyo Chemical Industry Co., Ltd.) was used instead of 4-bromobutyric acid ethyl ester (2.31 g, 0.01 mmol, Tokyo Chemical Industry Co., Ltd.) in Example 5-1. FIG. 9 shows a ¹H-NMR (CH₃OH-d) chart of Poly(VpyC₃C)40 thus obtained. m:n=3:2 from NMR signals.

### (Example 5-3) Poly(VpyC₃C)30

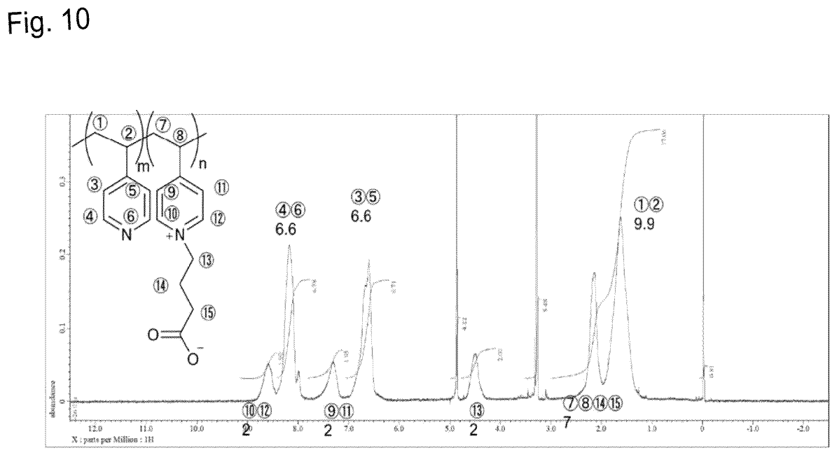

2.79 g of Poly(VpyC₃C)30 was obtained by the same operation as in Example 5-1 except that 4-bromobutyric acid ethyl ester (0.97 g, 0.005 mmol, Tokyo Chemical Industry Co., Ltd.) was used instead of 4-bromobutyric acid ethyl ester (2.31 g, 0.01 mmol, Tokyo Chemical Industry Co., Ltd.) in Example 5-1. FIG. 10 shows a ¹H-NMR (CH3OH-d) chart of Poly(VpyC₃C)30 thus obtained. m:n=3.3:1 from NMR signals.

### (Example 5-4) Poly(VpyC₃C)20

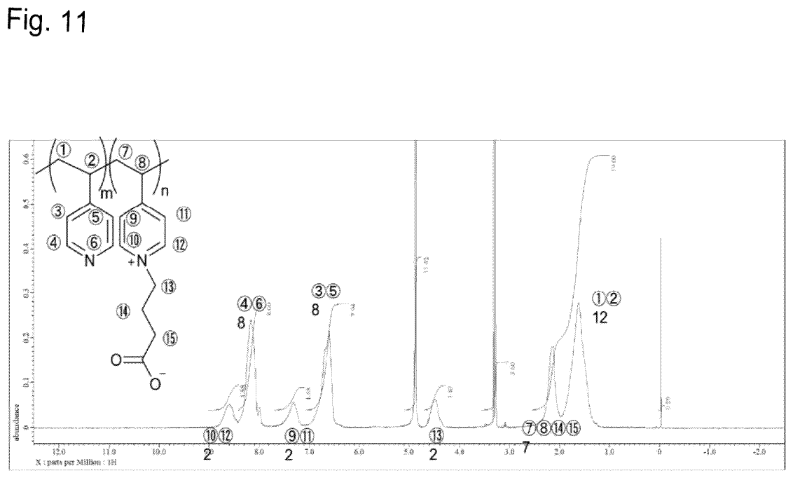

2.32 g of Poly(VpyC₃C)20 was obtained by the same operation as in Example 5-1 except that 4-bromobutyric acid ethyl ester (0.77 g, 0.003 mmol, Tokyo Chemical Industry Co., Ltd.) was used instead of 4-bromobutyric acid ethyl ester (2.31 g, 0.01 mmol, Tokyo Chemical Industry Co., Ltd.) in Example 5-1. FIG. 11 shows a ¹H-NMR (CH3OH-d) chart of Poly(VpyC₃C)20 thus obtained. m:n=4:1 from H-NMR signals.

### (Example 5-5) Poly(VpyC₃C)10

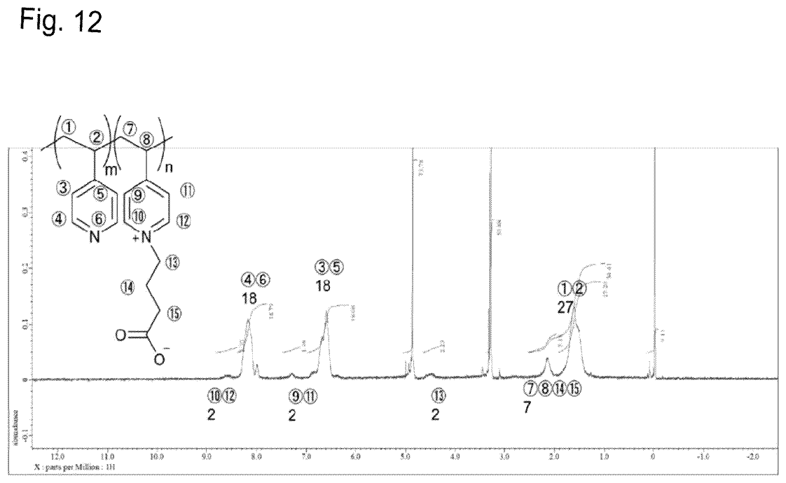

1.95 g of Poly(VpyC₃C)10 was obtained by the same operation as in Example 5-1 except that 4-bromobutyric acid ethyl ester (0.48 g, 0.001 mmol, Tokyo Chemical Industry Co., Ltd.) was used instead of 4-bromobutyric acid ethyl ester (2.31 g, 0.01 mmol, Tokyo Chemical Industry Co., Ltd.) in Example 5-1. FIG. 12 shows a ¹H-NMR. (CH3OH-d) chart of Poly(VpyC₃C)10 thus obtained. m:n=9:1 from ¹H-NMR signals.

### Example 6-1

### Production of Poly(VpyC₃S)40

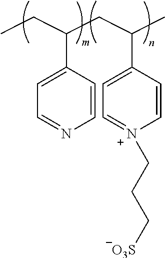

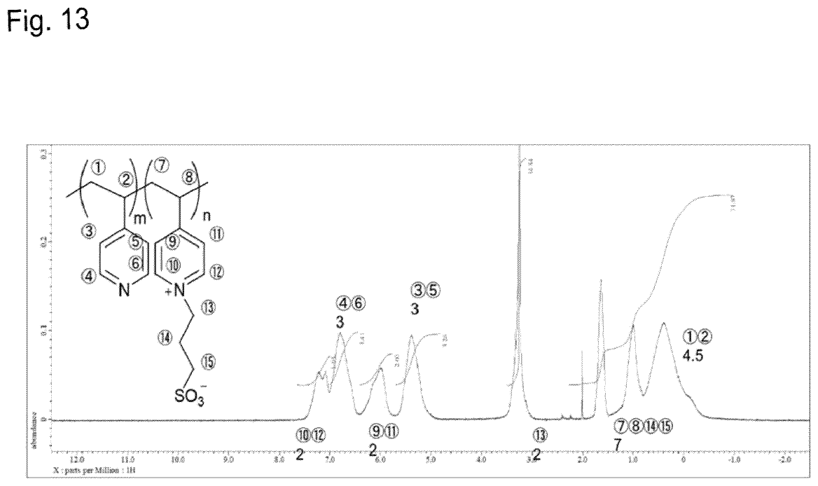

To a 2% NaCl solution of water:methanol (10 mL: 10 mL), poly(4-vinylpyridine) (Mw: 160000, Merck Japan K.K.) (1.97 g, 0.04 mmol) was dissolved in dichloromethane. To the solution, 1,3-propanesultone (0.84 g 0.006 mmol, Tokyo Chemical Industry Co., Ltd.) was added, and the mixture was reacted at 25° C. for 24 hours. The reaction solution was treated with a large amount of water for reprecipitation. The precipitates were freeze-dried to obtain Poly(VpyC₃S)40 (2.05 g). FIG. 13 shows a 1H-NMR (methanol-NaCl) chart of the product. The signal of a proton (signal 13 in FIG. 13) of a methylene group bonded to pyridine overlapped with the signal of water and thus could not be confirmed. Therefore, measurement was performed by warming to 50° C. As a result, a signal appeared at 4.9 ppm. m:n=3:2 from 1H-NMR signals.

### Example 6-2

Poly(VpyC₃S)15 was obtained by the same reaction as in Example 6-1 except that 1,3-propanesultone (0.23 g, 0.002 mmol, Tokyo Chemical Industry Co., Ltd.) was used instead of 1,3-propanesultone (0.84 g, 0.006 mmol, Tokyo Chemical Industry Co., Ltd.) in Example 6-1. m:n=17:3 from ¹H-NMR (CH₃OH—NaCl) signals.

### Example 7

### Production of Poly(VimC3C-co-C8Vim)

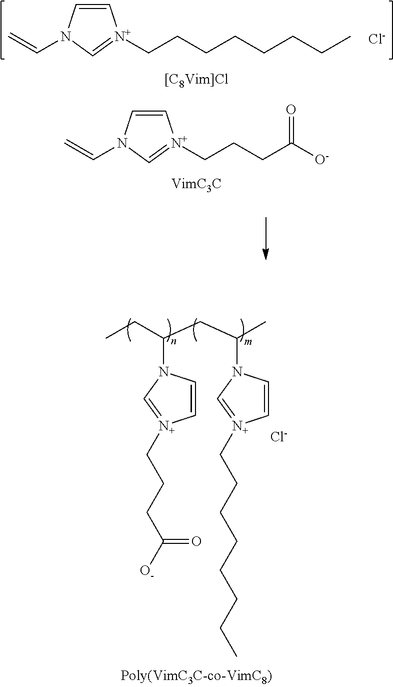

To 1 g of VimC₃C and [C₃Vim]Cl (charged ratio m:n=1:1) (1.375 M), 4 mL of methanol was added to dissolve these monomers. To the solution, 10 mol % of 2,2′-azobis [2-(2-imidazolin-2-yl)propane]dihydrochloride was added, and the mixture was sonicated for 180 minutes, followed by nitrogen substitution, and stirring for 17 hours in an oil bath of 60° C.

100 mL of DMSO (poor solvent) was stirred while cooled in ice water. The polymerization reaction solution dissolved in methanol was dropwise added thereto. The mixture was stirred until the polymerization reaction solution was solidified in the poor solvent. The reaction mixture was filtered through a membrane filter under reduced pressure. Finally, DMSO and the monomers in the copolymer were washed off with acetone, and the copolymer was recovered from the membrane filter. The recovered copolymer was stirred in a large amount of chloroform to remove DMSO.

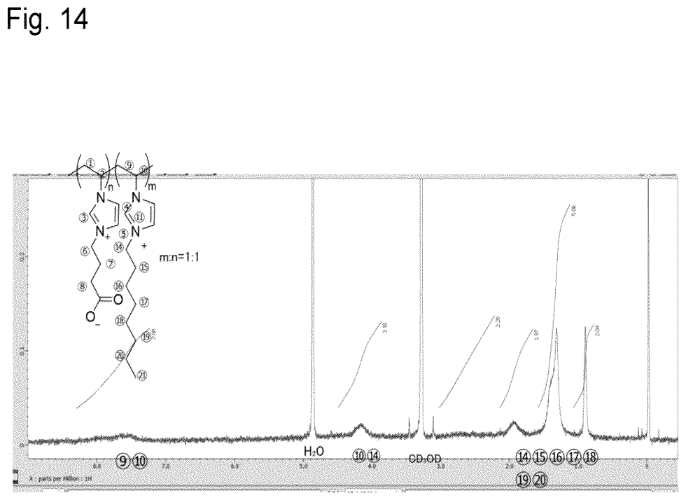

FIG. 14 shows a 1H-NMR (CD₃OD) chart of Poly(VimC3C-co-C8Vim) thus obtained.

### Example 8

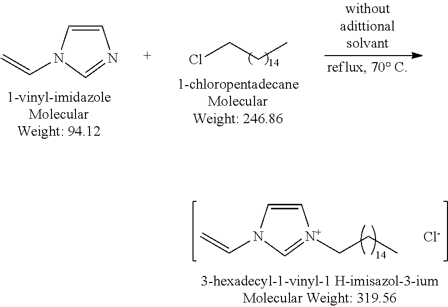

1-Vinyl-imidazole and pentadecane chloride were refluxed at 70° C. without a solvent to obtain [C16Vim]Cl.

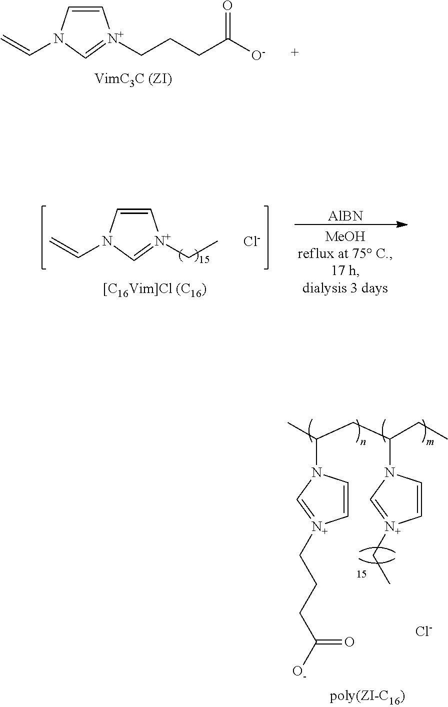

### (i) Production of Poly(VimC3C-co-C16Vim)-1.0

To 1 g of VimC₃C and [C16Vim]Cl (charged ratio m:n=1:1) (1.375 M), 4 mL of methanol was added to dissolve these monomers. To the solution, 10 mol % of 2,2′-azobis [2-(2-imidazolin-2-yl) propane]dihydrochloride was added, and the mixture was sonicated for 180 minutes, followed by nitrogen substitution, and refluxing for 17 hours in an oil bath of 75° C. Then, the reaction mixture was dialyzed for 3 days and purified to obtain poly(ZI-C16) (abbreviated to “(i) C16P”). The ratio of the purified polymer to the C16Vim unit determined by 1H-NMR was 0.34 molo, and the molecular weight was 84 kDa. The results are shown in Table 5.

### (ii) Production of Poly(VimC3C-co-C16Vim)-0.5

Poly(ZI-C16) (abbreviated to (ii) C16P) was obtained by the same operation as in the (i) C16P synthesis except that the charged ratio of 1 was changed to 1:0.5. The proportion of the C16Vim unit in the purified polymer determined by ¹H-NMR was 0.21 mol %, and the molecular weight was 374 kDa. The results are shown in Table 5.

(iii) Production of Poly(VimC3C-co-C16Vim)-0.1

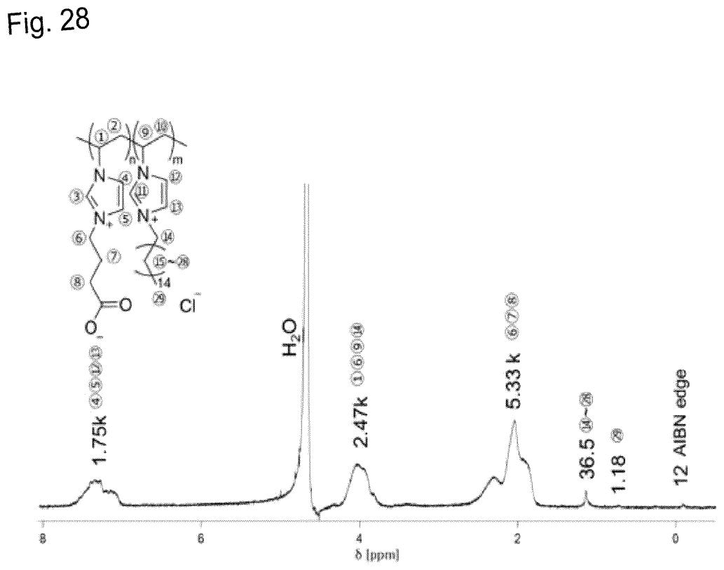

Poly(ZI-C16) (abbreviated to (iii) C16P) was obtained by the same operation as in the (i) C16P synthesis except that the charged ratio of 1 was changed to 1:0.1. The proportion of the C16Vim unit in the he purified polymer determined by ¹H-NMR was 0.05 mol %, and the molecular weight was 178 kDa. The results are shown in Table 5. FIG. 28 shows a ¹H-NMR chart.

### Example 9

VimC₃C (zwitterion monomer) and Poly(VimC₃C) (zwitterion polymer) produced in Example 1 were dissolved in water to prepare respective 10 mass& preservative solutions. The survival rate was determined using BOSC cells. The osmotic pressure of the polymer and the survival rate of the cells after cryopreservation were also determined. The results are shown in Table 1.

The survival rate of the aqueous zwitterion polymer solution was inferior to that of the aqueous zwitterion monomer solution. The cells preserved in the aqueous zwitterion polymer solution were observed to swell by the absorption of water and die. As the above reason, it is considered that when tried to conserve the cells using only the aqueous polymer solution, the solution became a hypotonic solution with a low osmotic pressure due to the increased molecular weight although the weight was the same. Accordingly, for adjusting the osmotic pressure, NaCl was added to the aqueous polymer solution, and the osmotic pressure was measured. The results are shown in Table 2. It is thereby evident that by adding 2% NaCl, the osmotic pressure equivalent to that of 10 mass % of the zwitterion monomer can be realized. As shown later in Example 10, a high cell survival rate was obtained by using a medium having an improved osmotic pressure.

TABLE 1

10 wt %

| | Polymer | Monomer |
|---|---|---|
| | Water | Water |
| Osmotic pressure | 5.3 | 860 |
| (mOsm/kg) | | |
| Survival rate | 0.2 | 0.15 |
| (BOSC) | | |
| Survival rate | 0.02 | 0.42 |
| (mNF) | | |

TABLE 2

| | Osmotic pressure |
|---|---|
| 10% Poly(VimC₃C) 0.5% NaClaq. | 149 |
| 10% Poly(VimC₃C) 1% NaClaq. | 289 |
| 10% Poly(VimC₃C) 2% NaClaq. | 965 (653) |

### Example 10

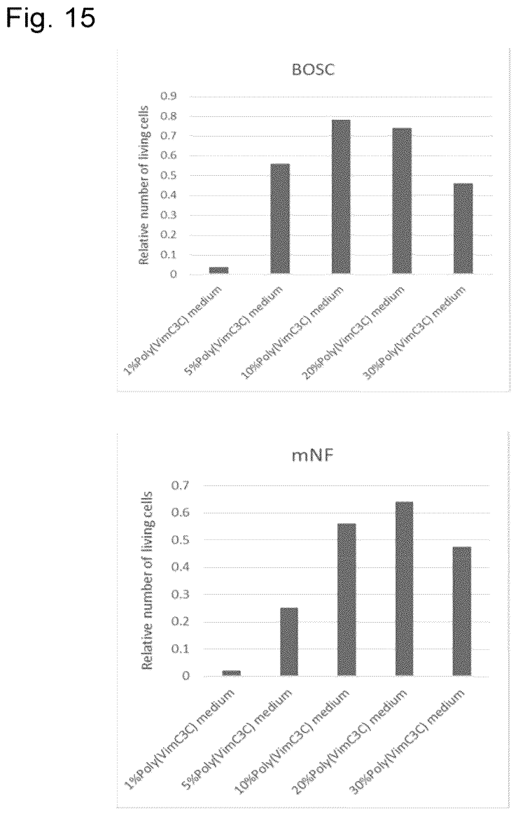

In order to study the relationship between a polymer concentration and a survival rate after cell cryopreservation, survival rates of cells (BOSC and mNF) after cryopreservation were measured when solutions of the Poly(VimC3C) polymer in DMEM (1 mass %, 5 mass %, 10 mass %, 20 mass %, and 30 mass %) were used as cryopreservative solutions. The results are shown in FIG. 15.

The cell survival rate was elevated with elevation in polymer concentration, and a maximum value was obtained at 10 to 20 mass %. This indicates that the polymer concentration has an optimum value.

### Example 11

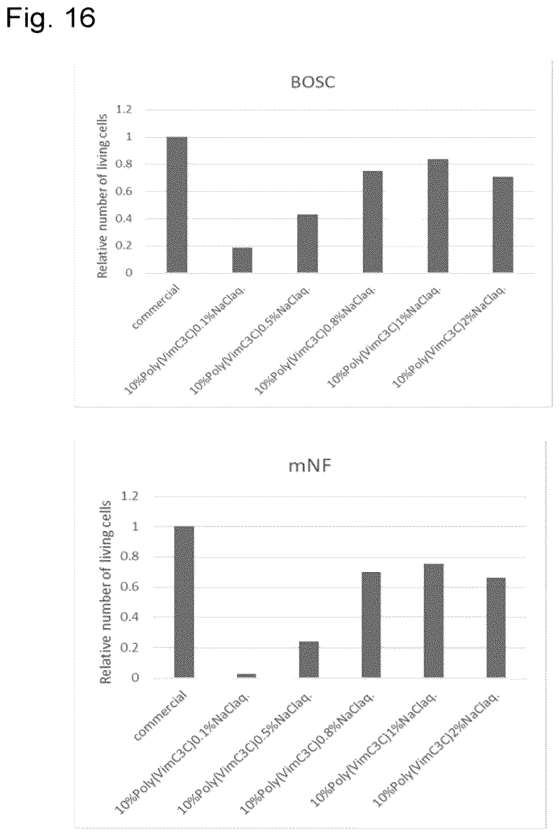

In order to study a survival rate after cell cryopreservation when an osmotic pressure was adjusted by the addition of NaCl to a polymer, solutions obtained by the addition of NaCl (0.1 mass %, 0.5 mass %, 0.8 mass %, 1 mass %, and 2 mass %) to a 10% aqueous Poly(VimC3C) polymer solution were used as cell cryopreservative solutions, and survival rates of cells (BOSC and mNF) after cryopreservation were measured. The results are shown in FIG. 16.

An optimum value of the NaCl concentration was approximately 1 mass %, indicating that an isotonic solution is most effective for cells. This suggests that the preservative solution may also be used as a cell culture solution.

### Example 12

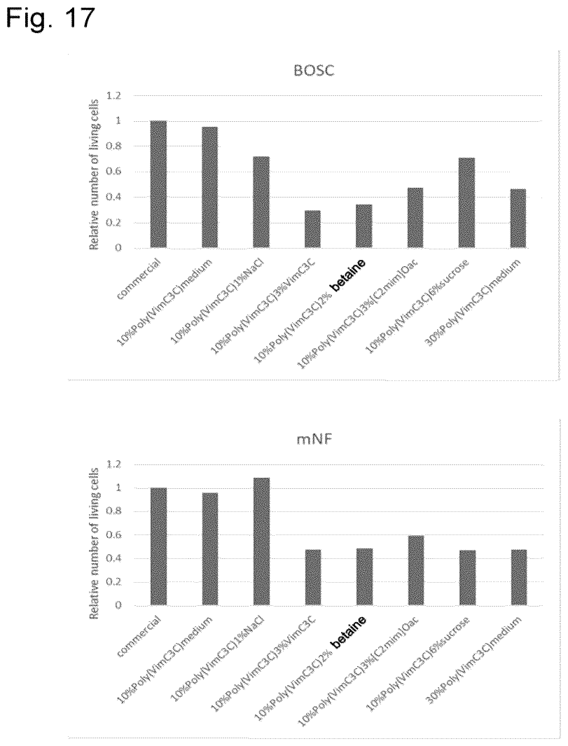

In order to study the type of a solute for osmotic pressure adjustment, sucrose (6% aqueous solution), trimethylglycine (2% aqueous solution), VimC₃C (3% aqueous solution of the monomer), and [C₂mim]OAc (3% aqueous solution of the monomer) were prepared and added in a predetermined amount to a 10% Poly(VimC₃C) solution to prepare cryopreservative solutions. Survival rates of cells (BOSC and mNF) after cryopreservation were measured. Table 3 and FIG. 17 show the relationship between the osmotic pressure and the cell survival rate. The cryopreservative solution containing the zwitterion monomer added had a low osmotic pressure, and the cell survival rate was not improved, probably due to the small amount added.

TABLE 3
2% NaCl aq.: 653 mOsm

| | Osmotic | Survival | Survival |
|---|---|---|---|
| | pressure | rate | rate |
| | (mOsm) | (BOSC) | (mNF) |
| 10% Poly(VimC₃C) PBS | 254 | 0.52 | 0.93 |
| 10% Poly(VimC₃C) medium | 272 | 0.96 | 0.96 |
| 10% Poly(VimC₃C) 1% NaClaq. | 289 | 0.72 | 1.08 |
| 10% Poly(VimC₃C) 6% sucroseaq. | 157 | 0.71 | 0.47 |
| 10% Poly(VimC₃C) 3% VimC₃Caq. | 165 | 0.3 | 0.47 |
| 10% Poly(VimC₃C) 2% trimethylglycineaq. | 177 | 0.34 | 0.48 |
| 10% Poly(VimC₃C) 3%[C₂mim]OAcaq. | 253 | 0.47 | 0.59 |

### Example 13

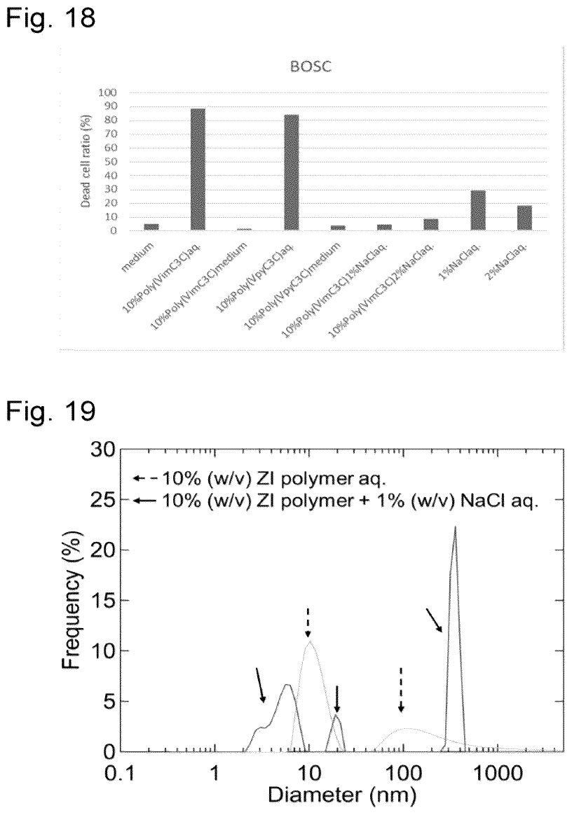

In order to study the cytotoxicity of a polymer, cells (BOSC) were left to stand in a medium containing the polymer. The results are shown in FIG. 18. It is evident that the solution containing the zwitterion polymer of the present invention has lower cytotoxicity than that of solutions containing other substances, which suggests that cells can be cultured in such a polymer-containing medium. Further, the dead cell ratio is lower than that of media in which the polymer is not added, which suggesting that adherent cells may be cultured in a floating state. The low dead cell ratio in the polymer solution was presumably because the cells were covered with a matrix of the polymer in a dispersed state ascribable to the electrolyte or because the cells adhere to each other via the matrix, thereby preventing cell death at the time of freezing and thawing. This result also suggests that this polymer may also act as a cell adhesive through the matrix formed by the polymer in a dispersed state ascribable to the electrolyte.

### Example 14

In order to study the influence of electrolyte addition to a polymer on the ice crystal formation of a solution, the polymer solution was measured by DSC. Table 4 shows DSC of a NaCl solution of the polymer and the proportion of ice crystals at the time of cryopreservation. As a result, it is evident that the NaCl solution of the polymer interacts with water.

TABLE 4

| | | Ratio | |
|---|---|---|---|
| | Melting | of ice | |
| | point | crystal | Glassifi- |
| | (° C.) | (%) | cation |
| 10% 1 mol % Poly(VimC3C)aq. | −0.8 | 108 | |
| 10% 10 mol % Poly(VimC3C)aq. | −1.6 | 99 | |
| 10% 1 mol % Poly(VimC3C)medium | −7.4 | 91 | −44° C. |
| 10% VimC3C10% 1 mol % | −5.4 | 93 | −86° C. |
| Poly(VimC3C)aq. | | | |
| 10% 1 mol % Poly(VimC3C)15% | −17 | 54 | |
| DMSOaq. | | | |
| 10% 1 mol % Poly(VimC3C)0.1% | −6.6 | 102 | |
| NaClaq. | | | |
| 10% 1 mol % Poly(VimC3C)0.5% | −5.5 | 113 | |
| NaClaq. | | | |
| 10% 1 mol % Poly(VimC3C)0.8% | −7.5 | 99 | |
| NaClaq. | | | |
| 10% 1 mol % Poly(VimC3C)1% NaClaq. | −4.9 | 95 | |
| 10% 1 mol % Poly(VimC3C)2% NaClaq. | −9.3 | 88 | |
| 10% 1 mol % Poly(VimC3C)PBS | −4.0 | 96 | |
| 2% NaCl aq. | −7.3 | 96 | |
| medium | −8.6 | 98 | −28° C. |

### Example 15

In order to study the influence of electrolyte addition to a polymer on aggregation, the polymer solution was measured by DSC.

FIG. 19 shows a DLS chart of the NaCl solution of the polymer. It is evident from FIG. 19 that the peak of the polymer shifts to the left as the electrolyte concentration is increased, and the addition of the electrolyte prevents the aggregation of the polymer.

It is considered from the results of Table 4 and FIG. 19 that the zwitterion polymer of the present invention became a dispersed state due to the electrolyte and interacts with cell membranes, which prevents cell death due to free-thaw. It is considered that the cells were covered with a matrix of the polymer in a dispersed state due to the electrolyte, or the cells adhere to one another via the matrix, whereby the cell death at the time of freeze-thaw was prevented.

### Example 16

In order to study the influence of the difference in the molecular weight of the polymer on the survival rate after the cell cryopreservation, the survival rates of cells (BOSC and mNF) after cryopreservation were measured when 10% Poly(VimC₃C) solutions differing in the molecular weight were used as the cryopreservative solutions.

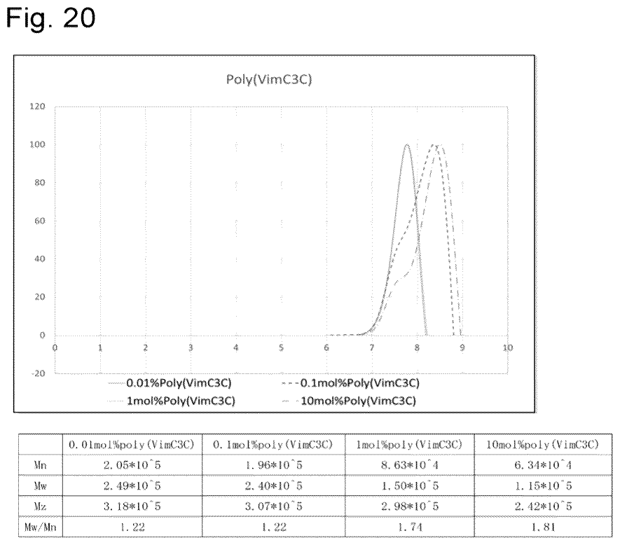

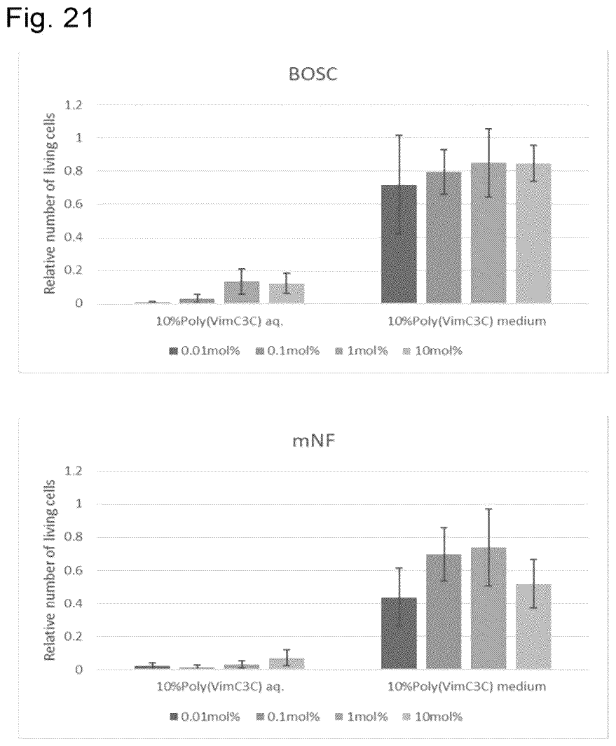

FIG. 20 shows GPC results of the polymer. FIG. 21 shows the relationship between the polymer and a cell survival rate. As shown in FIG. 20, the molecular weight of the polymer tended to decrease with the increase in the amount of the polymerization initiator.

As shown in FIG. 21, the amount of the polymerization initiator within the range of monomer ratios of from 0.1 mol % to 10 mol % has no influence on the survival rate after the cell cryopreservation. It is evident that the polymer having a molecular weight in the ranges of at least 100000 to 250000 as Mw and 50000 to 200000 as Mn can be used for a cryopreservative solution.

### Example 17

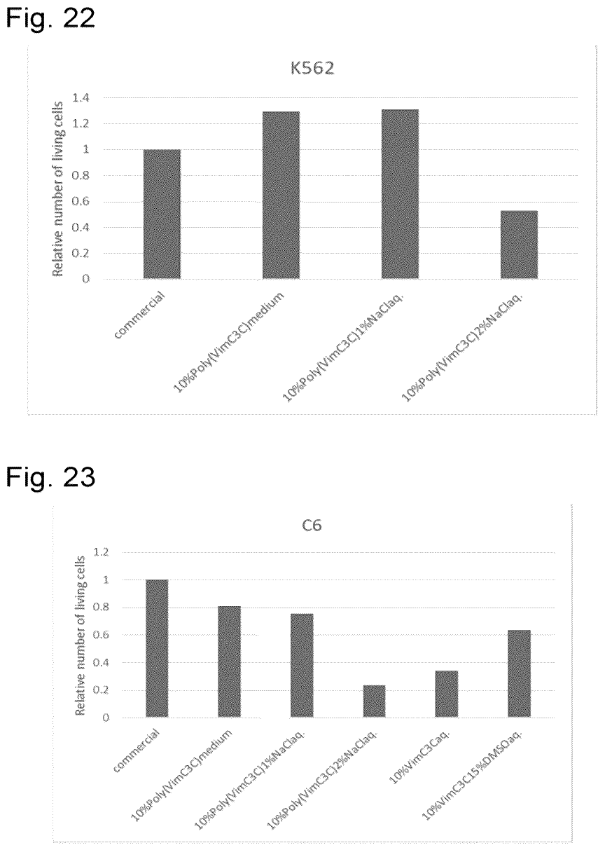

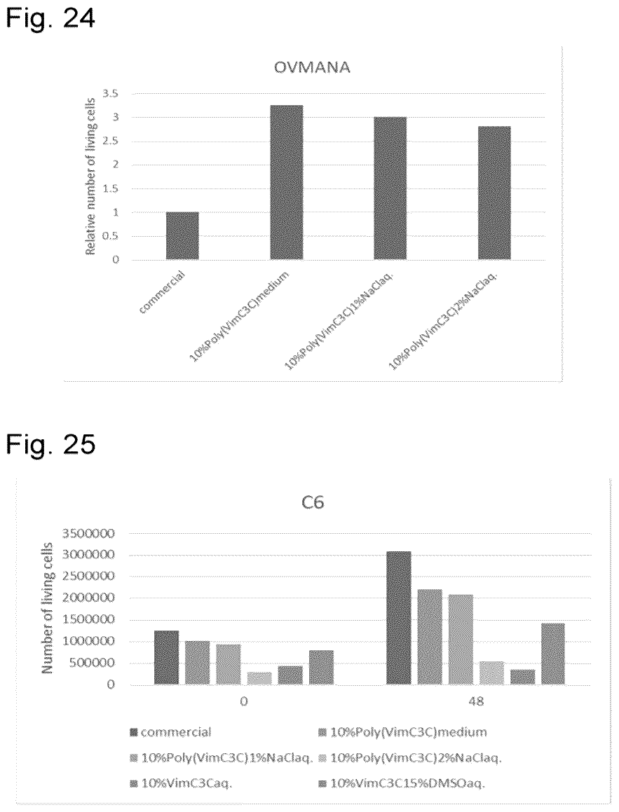

In order to study the influence of the polymer solution used as the cryopreservative solution on the survival rates of other cells after cryopreservation, the survival rates after cryopreservation were measured using three types of cells (K562 cells, C6 cells, and OVMANA cells). The results are shown in FIGS. 22, 23, and 24. It is evident that when the polymer of the present invention is used for a cryopreservative solution, the cell survival rate after cryopreservation is the same level as that of a commercially available product. Particularly, in the case of the K562 cells and the OVMANA cells considered vulnerable to cryopreservation the survival rates were higher than that in the case of using the commercially available cryopreservative solution.

### Example 18

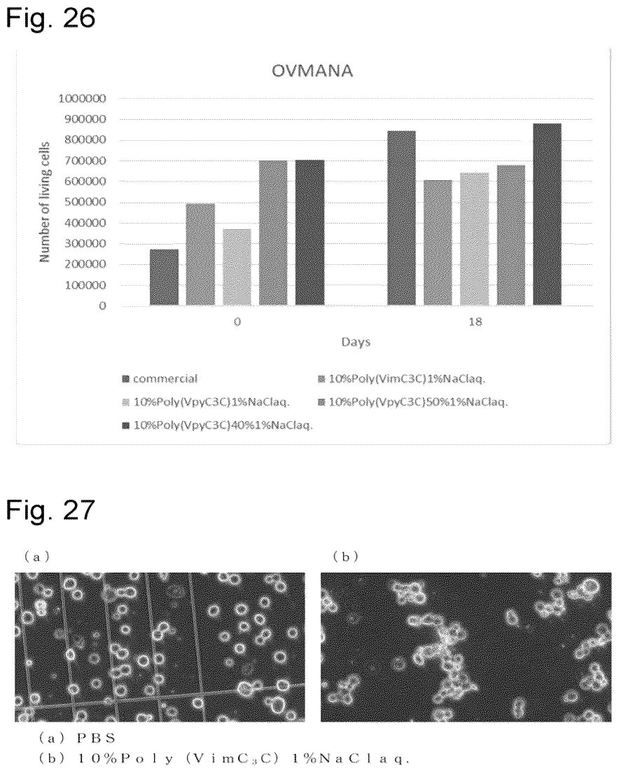

In order to study the influence of a polymer solution on a cell proliferation rate, cells (C6 cells and OVMANA cells) were cryopreserved and then cultured for 48 hours for the C6 cells and for 18 days for the OVMANA cells, and cells that proliferated were counted. The results are shown in FIGS. 25 and 26. The cells after cryopreservation using a solution containing the zwitterion polymer of the present invention was confirmed to proliferate. When the OVMANA cells are used, a larger number of cells was confirmed than that in a commercially available product. The C6 cells proliferated, though the results were slightly poorer than those of the commercially available product.

### Example 19

In order to observe cells in a polymer solution, the cells were left to stand at ordinary temperature for 60 minutes in PBS or a solution of 10% Poly(VimC₃C) and 1% NaCl. Results of microscopic observation are shown in FIG. 27. As a result, the cells in PBS (a) were evenly dispersed, whereas the cells in the polymer solution (b) adhered to each other. This suggested that the polymer forms a matrix in the vicinity of cells.

### Example 20

Poly(VimC3C-C16Vim) synthesized in Example 8 was examined for its cryopreserving effect.

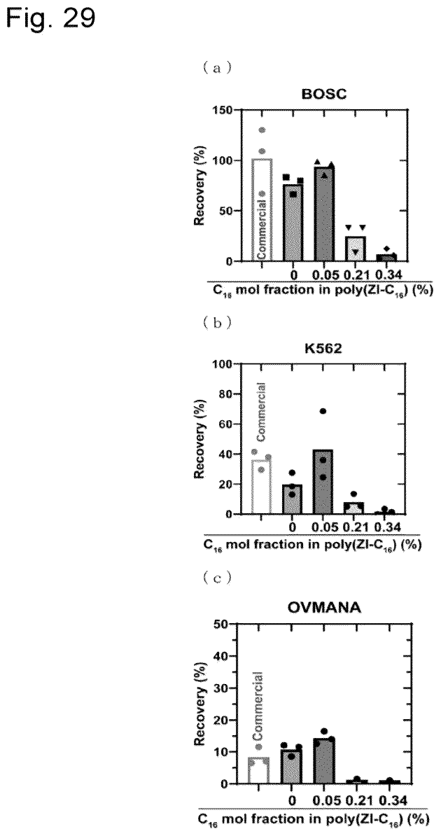

BOSC cells, K562 cells, and OVMANA cells were cryopreserved using 10 wt % of the poly(ZI-C16) polymer having a C16 monomer-derived unit content of 0.05 mol % (Example 8(iii)), 0.21 mol % (Example 8(ii)), or 0.34 mol % (Example 8(i)) in terms of the molar ratio, and the survival rates were examined. The polymer synthesized in Example 1 was used as the polymer having a C16 monomer-derived unit content of 0 mol %. The results are shown in FIG. 29. When the content of C16-derived unit was 0.05 mol %, favorable effects were obtained on all of the cell lines.

TABLE 5

| | (i) | (iii) | (iii) |
|---|---|---|---|
| Charged ratio of VimC3C and C16Vim | 1:1 | 1:0.5 | 1:0.1 |
| (molar ratio) | | | |
| Ratio of C16Vim to polymer | 0.34 | 0.21 | 0.05 |
| (mol %) | | | |
| Molecular weight estimated by 1H NMR | 84 | 374 | 178 |
| (kDa) | | | |

### Example 21

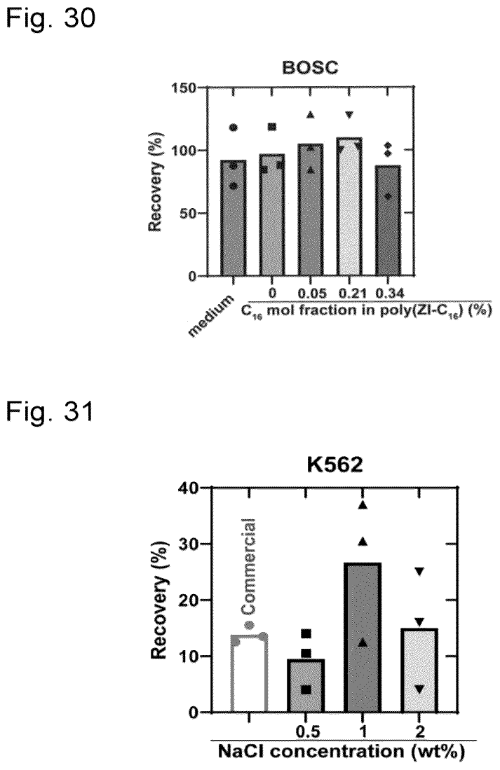

The toxicity of the poly(ZI-C16) synthesized in Example 8 to BOSC cells was examined. 1×10⁶ BOSC cells were incubated at 37° C. for 1 hour using a medium containing 10 wt % of the poly(ZI-C16) polymer having a C16 monomer-derived unit content of 0.05 mol % (Example 8(iii)), 0.21 mol % (Example 8(ii)), or 0.34 mol % (Example 8(i)) in terms of the molar ratio. Then, live cells were counted to determine the survival rate. The polymer synthesized in Example 1 was used as the polymer having a C16 monomer-derived unit content of 0 mol %. The results are shown in FIG. 30. C16 content-dependent toxicity was exhibited in none of (i) to (iii).

### Example 22

The influence of the salt concentration on the cryopreserving effect by the poly(ZI-C16) synthesized in Example 8 was examined.

A medium having a NaCl concentration of 0.5 wt %, 1 wt % or 2 wt % and containing 10 wt % of the polymer of Example 8 (iii) was prepared, K562 cells were cryopreserved, and the preservation rate was measured. The results are shown in FIG. 31. The solution having the NaCl concentration of 1 wt % was the most favorable solution.

### Example 23

(Synthesis of fluorescently labeled compound poly(ZI-C16-Fluorescein)

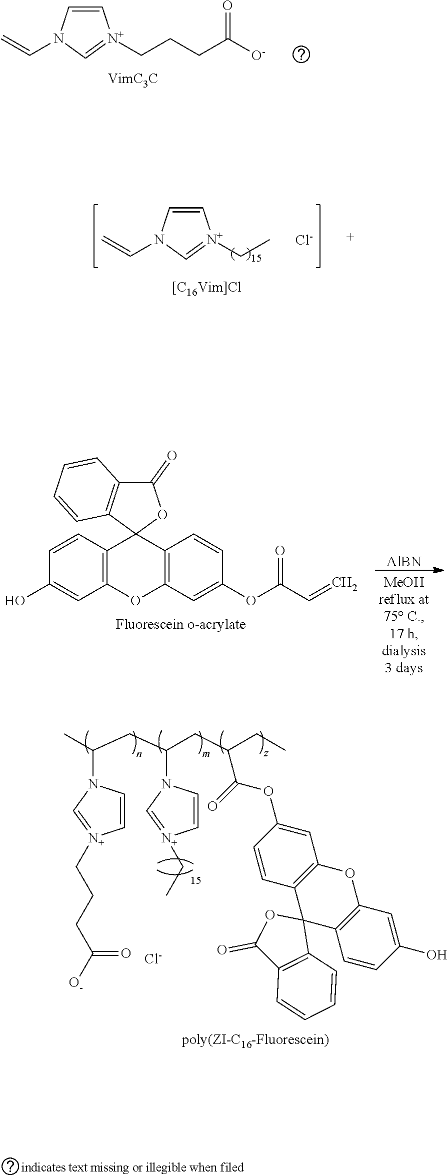

To MeOH, VimC3C, [C16Vim]Cl, and fluorescein o-acrylate were added at a molar ratio of 1:1:1, then AIBN was added, and the mixture was reacted at 75° C. for 17 hours, cooled, and then dialyzed for 3 days to obtain poly(ZI-C16-Fluorescein as a fluorescently labeled compound.

### Discussion about Mechanism

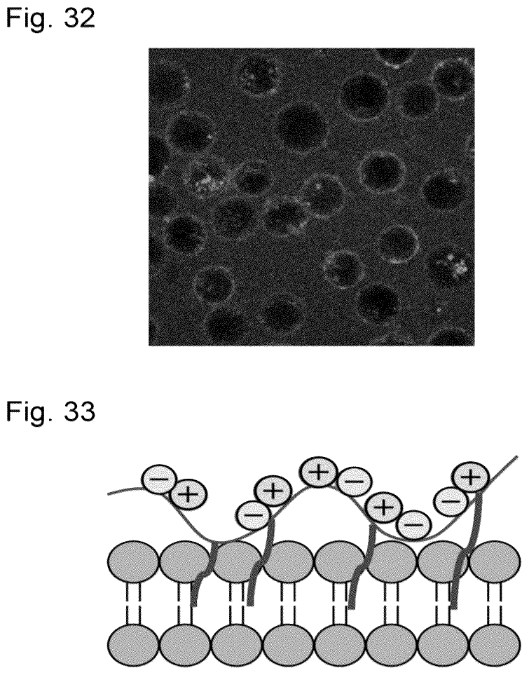

The synthesized fluorescent label was added to a medium of BOSC cells. 20 minutes later, the cells were washed with PBS and observed under a confocal microscope. The results are shown in FIG. 32. It is evident from FIG. 32 that poly(VimC3C-C16Vim) accumulated in the vicinity of cells, particularly, on cell membranes. It is considered that poly(VimC3C-C16Vim) forms a matrix outside cells and prevents the influx of ice crystals from outside the cells, particularly, on cell membranes. When an alkyl side chain of poly(VimC3C-C16Vim) was inserted into cell membranes from outside cells, it is considered that the polymer was anchored to the outside of the cell membranes and consequently prevented the influx of ice crystals so as to protect the cell membranes. The above is schematically explained in FIG. 33.

1. A zwitterion polymer of the following formula (1) or a labeled form thereof: 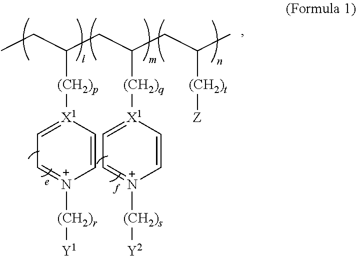
wherein
X¹ and X² are the same or different and each represent a carbon atom or a nitrogen atom;
Y¹ and Y² are the same or different and each represent an anion selected from the group consisting of —COO⁻, —SO₃⁻, —OP═O(H)O⁻, —OP═O(CH₃)O⁻, and —OP═O(OR¹)O⁻;
Z represents a hydrogen atom, an optionally alkyl group-substituted aromatic hydrocarbon group having 6 to 10 carbon atoms, an optionally alkyl group-substituted 5- or 6-membered aromatic heterocyclic group, an optionally alkyl group-substituted nitrogen-containing heterocyclic ammonium salt, a tetraalkylammonium salt, a tetraphenylphosphonium salt, a tetraalkylphosphonium salt, a trialkylsulfonium salt, or a linear or branched alkyl group having 1 to 22 carbon atoms and optionally having 1 to 3 oxygen atoms in a molecular chain;
e and f each represent an integer of 0 or 1;
l, m, and n are each a number which indicates a content ratio of each repeat unit and represent a number which satisfies 0<l≤1, 0≤m<1, 0≤n<1, and l+m+n=1; and
p, q, r, s, and t each represent an integer of 0 to 6.

2. The zwitterion polymer or the labeled form thereof of claim 1, wherein Y¹ and Y² are the same or different and are each an anion selected from the group consisting of —COO⁻ and —SO₃⁻.

3. The zwitterion polymer or the labeled form thereof of claim 1, wherein Z is at least one selected from the group consisting of an imidazolyl group, a pyridyl group, pyridinium chloride, C1 to C22 alkylpyridinium chloride, imidazolinium chloride, C1 to C22 alkylimidazolinium chloride, pyridinium bromide, C1 to C22 alkylpyridinium bromide, imidazolinium bromide, and C1 to C22 alkylimidazolinium bromide.

4. A preservative composition, comprising a zwitterion polymer of the following formula (1) or a labeled form thereof: 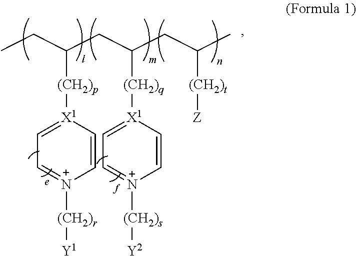
wherein
X¹ and X² are the same or different and each represent a carbon atom or a nitrogen atom;
Y¹ and Y² are the same or different and each represent an anion selected from the group consisting of —COO⁻, —SO₃⁻, —OP═O(H)O⁻, —OP═O(CH₃)O⁻, and —OP═O(OR¹)O⁻;
Z represents a hydrogen atom, an optionally alkyl group-substituted aromatic hydrocarbon group having 6 to 10 carbon atoms, an optionally alkyl group-substituted 5- or 6-membered aromatic heterocyclic group, an optionally alkyl group-substituted nitrogen-containing heterocyclic ammonium salt, a tetraalkylammonium salt, a tetraphenylphosphonium salt, a tetraalkylphosphonium salt, a trialkylsulfonium salt, or a linear or branched alkyl group having 1 to 22 carbon atoms and optionally having 1 to 3 oxygen atoms in a molecular chain;
e and f each represent an integer of 0 or 1;
l, m, and n are each a number which indicates a content ratio of each repeat unit and represent a number which satisfies 0<l≤1, 0≤m<1, 0≤n<1, and 1+m+n=1; and
p, q, r, s, and t each represent an integer of 0 to 6.

5. The preservative composition of claim 4, wherein Y¹ and Y² are the same or different and are each an anion selected from the group consisting of —COO⁻ and —SO₃⁻.

6. The preservative composition of claim 4, wherein Z is at least one selected from the group consisting of an imidazolyl group, a pyridyl group, pyridinium chloride, a C1-C22 alkylpyridinium chloride, imidazolinium chloride, a C1-C22 alkylimidazolinium chloride, pyridinium bromide, a C1-C22 alkylpyridinium bromide, imidazolinium bromide, and a C1-C22 alkylimidazolinium bromide.

7. The preservative composition of claim 4, further comprising one or more water-soluble compounds selected from the group consisting of an electrolyte, betaine, a zwitterion, an alcohol, a polyhydric alcohol, and a saccharide.

8. The preservative composition of claim 4, further comprising a cell-penetrating substance.

9. The preservative composition of claim 4, wherein the preservative composition is at least one selected from the group consisting of a cryopreservative composition for biological samples, a composition for culture of biological samples, a medium composition for biological samples, a composition for preservation of biological samples, a composition for functional maintenance of biological samples, and a composition for a functional test of biological samples.

10. A solubilizer, comprising the zwitterion polymer or a labeled form thereof of claim 1.

11. A method for preserving a biological sample, the method comprising bringing the biological sample into contact with a composition comprising the zwitterion polymer or the labeled form thereof of claim 1.

12. The method for preserving a biological sample of claim 11, wherein the composition comprising the zwitterion polymer or the labeled form thereof further comprises one or more water-soluble compounds selected from the group consisting of an electrolyte, betaine, a zwitterion, an alcohol, a polyhydric alcohol, and a saccharide.

13. The method for preserving a biological sample of claim 11, wherein the composition comprising the zwitterion polymer or the labeled form thereof further comprises a cell-penetrating substance.

14. The method for preserving a biological sample of claim 11, wherein the biological sample is preserved for at least one selected from the group consisting of cryopreservation of the biological sample, culture of the biological sample, a medium of the biological sample, functional maintenance of the biological sample, and a functional test of the biological sample.

15. A method for dissolving a poorly soluble substance, comprising dissolving the poorly soluble substance in a composition comprising the zwitterion polymer or the labeled form thereof of claim 1.
